(19) United States
(12) Patent Application Publication
(10) Pub. No.: US 2016/0206246 A1
(43) Pub. Date: Jul. 21, 2016

(54) OBJECT INFORMATION ACQUIRING APPARATUS AND OBJECT INFORMATION ACQUISITION METHOD

(71) Applicant: CANON KABUSHIKI KAISHA, Tokyo (JP)

(72) Inventors: Yoshitaka Baba, Tokyo (JP); Naoto Abe, Machida-shi (JP); Takao Nakajima, Kawasaki-shi (JP); Hiroshi Yamamoto, Kawasaki-shi (JP)

(21) Appl. No.: 14/993,206

(22) Filed: Jan. 12, 2016

(30) Foreign Application Priority Data
Jan. 20, 2015 (JP) 2015-008892

(51) Int. Cl. A61B 5/00

(52) U.S. Cl. CPC A61B 5/725; A61B 5/0095; A61B 5/7225; A61B 2560/0475

(57) ABSTRACT

An object information acquiring apparatus comprises an irradiation unit that irradiates an object with light; a receiving unit including detection elements that receives acoustic waves generated from the object and outputs analog signals; a conversion unit that performs A/D conversion on the analog signals to generate first digital signals; a memory that stores the first digital signals; a correction unit that generates second digital signals by correcting the first digital signals using different frequency filter in accordance with a positional relation between a position in which characteristic information of the object is acquired and each of the detection elements; and an acquiring unit that acquires the characteristic information of the object based on the second digital signals.

### BACKGROUND OF THE INVENTION

1. Field of the Invention

The present invention relates to an object information acquiring apparatus and an object information acquisition method.

2. Description of the Related Art

Researches on an optical imaging apparatus that irradiates an object such as a living body with light emitted from a light source such as a laser and visualizes information on the inside of the object obtained based on the incident light have progressed actively in a medical field. A photoacoustic imaging (PAI) technique is known as one of the optical imaging techniques. According to the photoacoustic imaging technique, an object is irradiated with pulsed beams generated from a light source, acoustic waves (typically, ultrasound waves) generated from the tissues of the object having absorbed the energy of the pulsed beams having propagated and diffused inside the object are received, and object information is imaged (visualized) based on the reception signals.

That is, a probe receives elastic waves (photoacoustic waves) generated when a subject segment absorbs the irradiated optical energy and expands instantaneously by taking advantage of the difference in the rate of absorption between a subject segment such as a tumor and the other tissues. By mathematically analyzing and processing the reception signals, it is possible to obtain information on the inside of the object (in particular, an initial acoustic pressure distribution, an optical energy density distribution, or an absorption coefficient distribution). These items of information can be used for quantitative measurements of a specific substance inside the object (e.g. oxygen saturation in the blood). In recent years, preclinical researches for imaging the blood vessel images of a small animal using the photoacoustic imaging technique and clinical researches for application of this principle to diagnosis of the breast cancers or the like have progressed actively.

For example, the specification of US Patent Application Publication No. 2011/0306865 discloses a photoacoustic imaging apparatus that performs photoacoustic imaging using a probe in which transducers are disposed on a hemisphere. According to this probe, photoacoustic waves generated in a specific area can be received with high sensitivity. Thus, the resolution of the object information in a specific area increases. Further, the specification of US Patent Application Publication No. 2011/0306865 discloses that the central frequency of the probe used for receiving acoustic waves is between 1 to 30 MHz. As described above, according to the photoacoustic imaging technique, it is known that the frequency range of acoustic waves generated inside an object is several tens of MHz.

Moreover, Japanese Patent Application Publication No. 2012-179348, for example, discloses a technique of scanning a probe in which transducers are disposed on a hemisphere within a certain plane, moving the probe in a direction vertical to the scanning plane to scan the probe in another plane, and performing such a scanning operation a plurality of number of times.

According to the scanning method disclosed in Japanese Patent Application Publication No. 2012-179348, it is possible to obtain object information with high resolution in a wide range of areas when a probe in which transducers are disposed on a hemisphere is used.

### SUMMARY OF THE INVENTION

The distance from a certain originating point of acoustic waves in an object to a plurality of acoustic wave detection elements arranged in a probe is different from one acoustic wave detection element to another. Thus, acoustic waves generated at a certain point in the object reach respective acoustic wave detection elements while being attenuated by a degree corresponding to a propagation distance that the acoustic waves travel until reaching the plurality of acoustic wave detection elements. Further, the degree of attenuation of acoustic waves depends on the frequency characteristics of the generated acoustic waves.

In view of the above problems, it is an object of the present invention to provide an object information acquiring apparatus capable of enabling acoustic waves in a wide frequency range to be used for image formation.

The present invention in its one aspect provides an object information acquiring apparatus comprising an irradiation unit configured to irradiate an object with light; a receiving unit including a plurality of acoustic wave detection elements configured to receive acoustic waves generated from the object by irradiation with the light by the irradiation unit, and to output analog signals; a conversion unit configured to perform analog-to-digital conversion on the analog signals output from the plurality of acoustic wave detection elements to generate a plurality of first digital signals; a memory configured to store the plurality of first digital signals; a correction unit configured to generate a plurality of second digital signals by correcting the plurality of first digital signals stored in the memory using a different frequency filter in accordance with a positional relation between a position in which characteristic information of the object is acquired and each of the plurality of acoustic wave detection elements; and an acquiring unit configured to acquire the characteristic information of the object based on the plurality of second digital signals.

The present invention in its another aspect provides an object information acquisition method for acquiring characteristic information of an object based on a plurality of first signals obtained and stored by receiving, using a plurality of acoustic wave detection elements, acoustic waves generated from the object when irradiated with light, the method comprising a first step of generating a plurality of second signals by correcting the plurality of first signals using a different frequency filter in accordance with a positional relation between a position in which characteristic information of the object is acquired and each of the plurality of acoustic wave detection elements; and a second step of acquiring the characteristic information of the object based on the plurality of second signals.

The present invention in its another aspect provides a non-transitory computer readable storing medium recording a computer program for causing a computer to perform the object information acquisition method.

According to the present invention, it is possible to provide an object information acquiring apparatus capable of enabling acoustic waves in a wide frequency range to be used for image formation.

Further features of the present invention will become apparent from the following description of exemplary embodiments with reference to the attached drawings.

### BRIEF DESCRIPTION OF THE DRAWINGS

FIG. 1A is a block diagram illustrating Example 1 of an object information acquiring apparatus of the present invention;

FIG. 1B is a block diagram illustrating a probe unit of Example 1;

FIG. 1C is a block diagram illustrating a data acquisition unit according to Example 1;

FIGS. 2A to 2C are cross-sectional views illustrating a light intensity distribution inside an object according to Example 1;

FIG. 3 is a diagram illustrating the relation between a depth inside an object and an intensity of acoustic waves according to Example 1;

FIGS. 4A to 4D are diagrams illustrating the relation between an arrangement of reception elements and the obtained signal intensity according to Example 1;

FIG. 5 is a diagram illustrating the state during photoacoustic measurement according to Example 1;

FIGS. 6A and 6B are diagrams illustrating the relation between the light intensity and the depth during photoacoustic measurement according to Example 1;

FIG. 7 is a diagram illustrating the state during another photoacoustic measurement according to Example 1;

FIGS. 8A and 8B are diagrams illustrating the relation between the light intensity and the depth during another photoacoustic measurement according to Example 1;

FIG. 9 is a diagram illustrating the relation between the digital gain and the time according to Example 1;

FIGS. 10A and 10B are diagrams illustrating a data storage state of a memory according to Example 1;

FIG. 11 is a diagram illustrating an acquisition state of digital signals in a memory according to Example 1;

FIG. 12 is a flowchart illustrating the function of a data acquisition unit according to Example 1;

FIG. 13A is a diagram illustrating Example 2 of the object information acquiring apparatus of the present invention;

FIG. 13B is a diagram illustrating the relation between the sampling time and the digital gain pattern according to Example 2;

FIG. 14A is a diagram illustrating Example 3 of the object information acquiring apparatus of the present invention;

FIG. 14B is a diagram illustrating a measurement state at another position of a receiving unit according to Example 3;

FIGS. 15A to 15D are diagrams illustrating the relation between the time and the digital gain pattern according to Example 3;

FIG. 16 is a diagram illustrating Example 4 of the object information acquiring apparatus of the present invention;

FIG. 17 is a diagram illustrating another measurement state according to Example 4; and

FIGS. 18A and 18B are diagrams illustrating a light intensity profile according to Example 4.

### DESCRIPTION OF THE EMBODIMENTS

Hereinafter, embodiments of the present invention will be described with reference to the drawings. As a general rule, the same constituent elements will be denoted by the same reference numerals, and the description thereof will be omitted. Detailed calculation formula, calculation procedures, and the like described below are to be appropriately changed according to the configuration and various conditions of an apparatus to which the present invention is applied, and the scope of the present invention is not limited to those described below.

An object information acquiring apparatus of the present invention includes an apparatus which uses a photoacoustic effect to receive acoustic waves generated inside an object by irradiating the object with light (electromagnetic waves) such as near-infrared light to acquire object information as image data.

In the case of an apparatus which uses a photoacoustic effect, the acquired object information indicates a generation source distribution of acoustic waves generated by light irradiation, an initial acoustic pressure distribution inside the object, an optical energy absorption density distribution and an absorption coefficient distribution derived from the initial acoustic pressure distribution, or a concentration distribution of a substance that constitutes a tissue. Examples of the substance concentration distribution include an oxygen saturation distribution, a total hemoglobin concentration distribution, and an oxygenated or reduced hemoglobin concentration distribution.

Moreover, characteristic information which is the object information at a plurality of positions may be acquired as a 2-dimensional or 3-dimensional characteristics distribution. The characteristics distribution is generated as image data indicating the characteristic information inside an object.

The acoustic wave referred in the present invention is typically an ultrasound wave and includes an elastic wave called a sound wave and an acoustic wave. The acoustic wave generated by the photoacoustic effect is referred to as a photoacoustic wave or a light-induced ultrasound wave. An acoustic wave detection element (e.g. a piezoelectric element) receives acoustic waves generated inside or reflected from an object. Further, the present invention can be understood as a control method when acoustic waves are received by the object information acquiring apparatus.

Hereinafter, an object information acquiring apparatus (a photoacoustic imaging apparatus) will be described. First, the relation between the acoustic pressure of acoustic waves generated by a photoacoustic effect and the light intensity inside an object will be described. The acoustic pressure p₀ [Pa] of acoustic waves generated by the photoacoustic effect is expressed by Equation (1) below.

p₀=μₐ*Γ*Φ (1)

In Equation (1), μₐ is an absorption coefficient (unit: /mm) of a light absorber (tumor or the like). Γ is a Gruneisen coefficient. Φ is the light intensity [J/mm²] at the position of the light absorber. The Gruneisen coefficient Γ is a division of the product of a volume expansion coefficient and the square of the acoustic velocity by the specific heat capacity and takes an approximately constant value when the object is a living body.

As can be understood from Equation (1), the generated acoustic pressure p₀ is proportional to the light intensity Φ. When the object is a strong light scattering object like a living body, the light intensity Φ decays exponentially according to the distance from the light irradiation position. Thus, the light intensity Φ decreases with attenuation and the acoustic pressure p₀ of the generated acoustic waves decreases. In this case, the frequency of acoustic waves generated at a certain point inside the object depends on the size of a light absorber which is the generation source of acoustic waves. It is known that the waveform of electrical signals obtained by acoustic wave detection elements converting acoustic waves to electrical signals (corresponding to analog signals) has an N-shape, and the frequency of the electrical signals is the reciprocal of a time width T of the N-shape. In the case of a spherical light absorber, the time width T can be calculated from a relational expression of T=d/c where d is the diameter of the light absorber and c is the acoustic velocity. For example, when the acoustic velocity is 1500 m/s, acoustic waves having a time width T of 1 μs (that is, a frequency of 1 MHz) are generated from the light absorber having a diameter of 1.5 mm.

Further, the generated acoustic waves propagate inside the object while being attenuated due to the influence of frequency-dependent attenuation (FDA). For example, the frequency-dependent attenuation in a normal breast is approximately 1.27 dB/(cm·MHz), and the larger the propagation distance of acoustic waves in the object and the higher the frequency, the more the acoustic waves decay. Further, distance-dependent attenuation caused by energy dissipation due to spherical wave propagation or cylindrical wave propagation may be taken into consideration. As described above, acoustic waves generated at a certain point inside an object with the light intensity attenuated as compared to the light intensity on the surface of the object reach reception elements with a certain acoustic pressure due to the influence of the distance-dependent attenuation caused by energy dissipation as well as the frequency-dependent attenuation. Acoustic waves having an acoustic pressure higher than a smallest reception acoustic pressure which is the smallest value of an acoustic pressure that a reception element can use substantially for image reconstruction at the time of reaching the reception element can contribute to visualization of the inside of the object and are acoustic waves which are of a great significance to acquire. The acoustic pressure that a reception element can use substantially for image reconstruction is an acoustic pressure having the magnitude of such a vibration amplitude that can output an electrical signal that the reception element can use substantially for image reconstruction. On the other hand, acoustic waves having an acoustic pressure lower than the smallest reception acoustic pressure of a reception element at the time of reaching the reception element are acoustic waves which do not contribute to visualization of the inside of the object or serve as noise components, and are of a small significance to acquire.

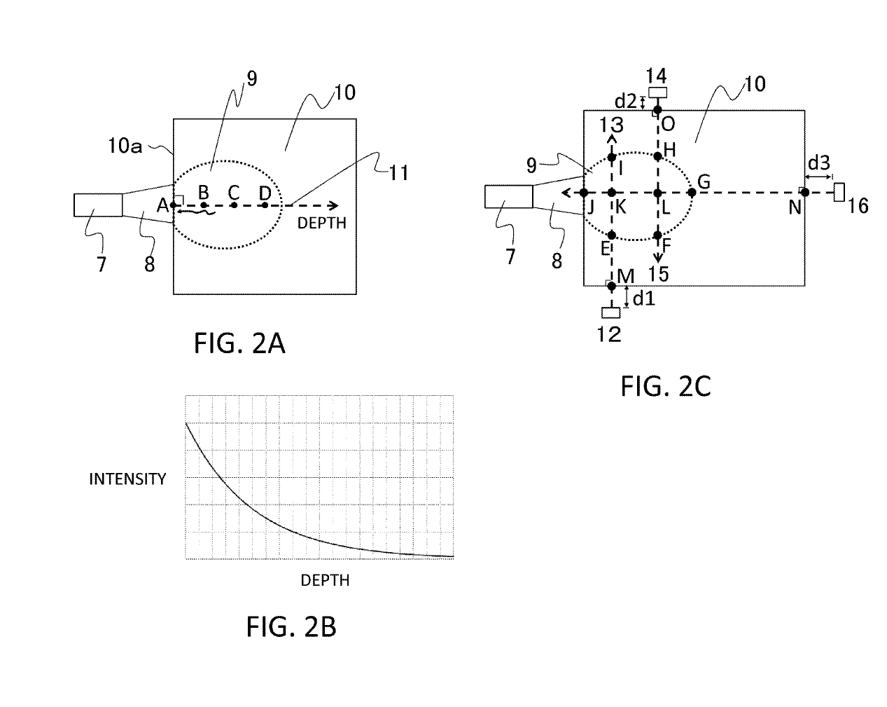

FIGS. 2A to 2C are cross-sectional views illustrating a light intensity distribution inside an object according to Example 1. FIG. 2A is a cross-sectional view illustrating a light intensity distribution when the object is irradiated with light which is a pulsed beam and illustrates a case in which an object 10 is irradiated with light 8 from a light source 7. An area 9 indicates a light intensity distribution (also referred to as a light intensity profile) inside the object 10. The light intensity is not uniformly distributed over the entire area 9, but practically, the light intensity distribution state may be different depending on the position in the area 9 due to the influence of the shape of the object 10 and the diffusion of pulsed beams inside the object 10. Here, points A, B, C, and D are points at the positions of the depths 0, 10, 20, and 30 mm along a normal line 11 of a point A on a surface 10a of the object 10.

FIG. 2B is a diagram illustrating the relation between the depth inside an object and the intensity of light. Here, it is assumed that the absorption coefficients of light absorbers (tumors or the like) present at the points B, C, and D are μ_{α}_{_}₁₀ and are equal to each other. As is obvious from FIG. 2B, the intensity of light decays exponentially as the depth increases. For example, acoustic waves generated at the point B has a smaller initial acoustic pressure than the acoustic waves generated at the point A because the intensity of irradiated light decays inside the object 10 as illustrated in FIG. 2B. Further, the acoustic waves generated at the point B decay due to the influence of the frequency-dependent attenuation until reaching the point A.

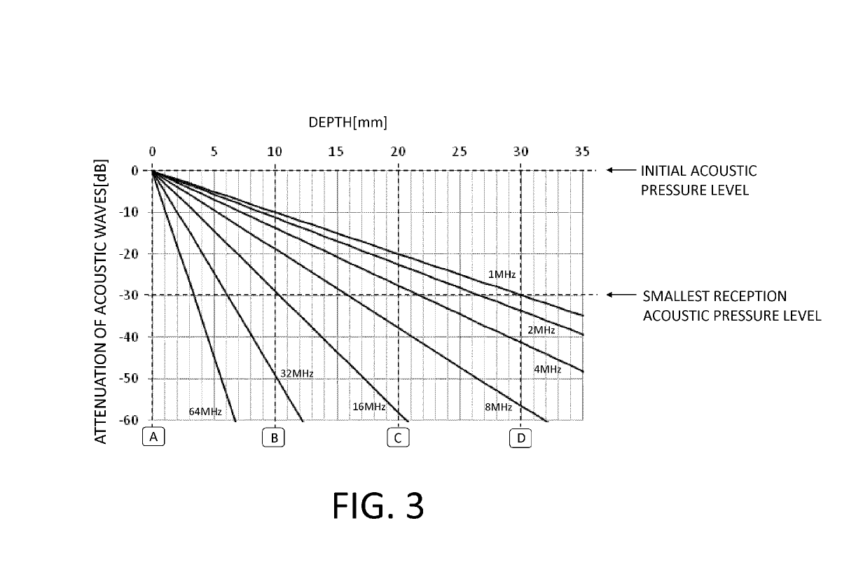

FIG. 3 is a diagram illustrating the relation between the depth inside an object and the intensity and the frequency of acoustic waves. Here, the points A to D correspond to those of FIG. 2B. FIG. 3 illustrates the degree of attenuation at each frequency, of acoustic waves generated from light absorbers assumed to be present at the respective points B to D and have propagated to the point A as compared to acoustic waves generates by a light absorber assumed to be present at the point A when acoustic waves are observed at the point A. In FIG. 3, for example, when acoustic waves having the frequency 1 MHz generated at the point B have propagated to the point A, the acoustic waves reach the point A in a state in which the acoustic pressure is lower by 10 dB than the initial acoustic pressure of acoustic waves having the frequency 1 MHz generated at the point A. Moreover, for example, acoustic waves having the frequency 16 MHz generated at the point B reach the point A in a state in which the acoustic pressure is lower by approximately 30 dB than the initial acoustic pressure of the acoustic waves having the frequency 16 MHz generated at the point A. Here, it is assumed that a reception element (not illustrated) disposed at the point A to observe acoustic waves can detect acoustic pressure which is 30 dB smaller than the initial acoustic pressure of acoustic waves generated by a light absorber present at the point A, having the absorption coefficient μ_{α}_{_}₁₀. In this case, the smallest reception acoustic pressure level (corresponding to −30 dB on the Y-axis) illustrated in FIG. 3 indicates the lower limit of an acoustic pressure detection level. In this way, the range of signal frequencies of acoustic waves generated by the light absorbers having the absorption coefficient μ_{α}_{_}₁₀ present at the points B to D that can be detected by the reception element at the point A can be read from FIG. 3. That is, if a signal belongs to such a detectable frequency range, the signal does not serve as noise when object information is acquired (that is, image reconstruction is performed) but object information can be acquired substantially. An extraction processing unit described later extracts the frequency range after converting the analog signal output by the reception element into a digital signal.

More specifically, a reception element at the point A can detect acoustic waves generated at the point B, having the frequency of up to approximately 16 MHz, acoustic waves generated at the point C, having the frequency of up to approximately 4 MHz, and acoustic waves generated at the point D, having the frequency of up to approximately 1 MHz. From this, when the absorption coefficient μ_{α}_{_}₁₀ of the light absorber in the object 10 is assumed and the smallest reception acoustic pressure of the reception element in the light intensity distribution area 9 in the object 10 are obvious, it is possible to predict the frequency range of acoustic waves that can reach the reception element from a point at a certain measurement depth in the object 10. Based on the predicted range, it is possible to determine the frequency of acoustic waves and the measurement depth at which the acoustic waves are acquired, which can satisfactorily contribute to visualization (image reconstruction) of the inside of the object. Moreover, a gain value (corresponding to a gain) necessary for correcting attenuation of the acoustic pressure of acoustic waves having predetermined frequency characteristics so that significant signals contributing to visualization can be acquired can be calculated based on the degree of frequency-dependent attenuation of the object 10 and the occurrence depth of acoustic waves. Moreover, a decrease and a variation in the initial acoustic pressure resulting from light intensity attenuation in the object 10 can be also corrected based on the information on the light intensity distribution area 9 in the object 10.

FIG. 3 illustrates the attenuation of acoustic waves taking the attenuation of irradiated light and the frequency-dependent attenuation in the object only into consideration in order to simplify the description. However, practically, distance-dependent attenuation caused by energy dissipation due to spherical wave propagation or cylindrical wave propagation needs to be taken into consideration. In this case, the degree of attenuation becomes larger than that illustrated in FIG. 3.

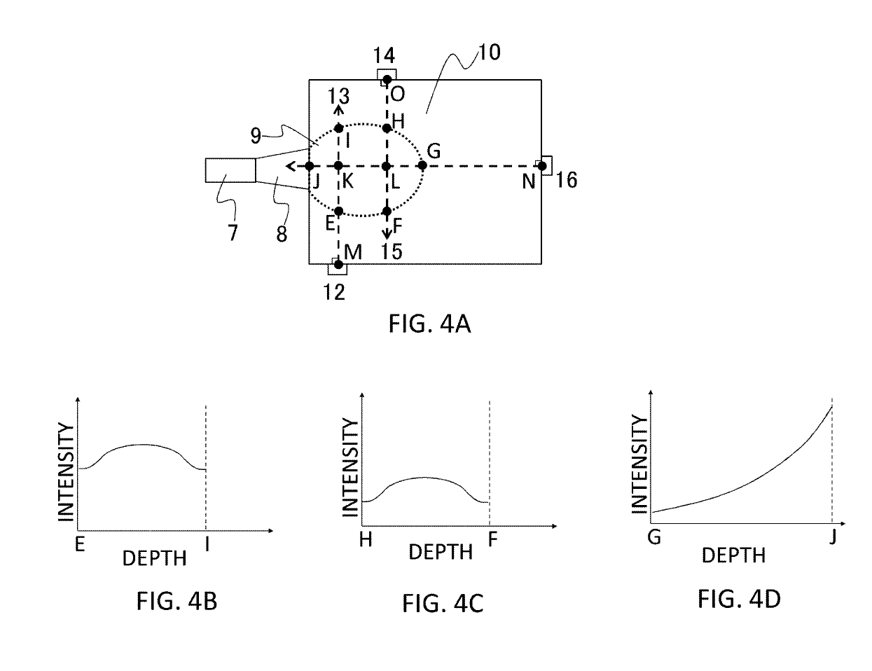

FIGS. 4A to 4D are diagrams illustrating the relation between the arrangement of reception elements and the obtained signal intensity according to Example 1. FIG. 4A is a cross-sectional view illustrating a state in which photoacoustic waves are detected by a plurality of reception elements. The reception elements 12, 14, and 16 are disposed in a distributed manner. That is, a case in which an object 10 is irradiated with light 8 from a light source 7 in this arrangement state and generated acoustic waves are detected by reception elements 12, 14, and 16 will be considered. It is assumed that the reception element receives acoustic waves generated from a point present in a normal direction of a point M. That is, the reception element 12 receives acoustic waves generated from a point on a segment EI, the reception element 14 receives acoustic waves generated from a point on a segment FH, and the reception element 16 receives acoustic waves generated from a point on a segment GJ. It is assumed that the reception element receives acoustic waves generated from a point present in a normal direction of a point O. It is assumed that the reception element 16 receives acoustic waves generated from a point present in a normal direction of a point N. Here, when acoustic waves generated with an initial acoustic pressure P₀ at the point K are received by the reception elements 12 and 16, the distance (corresponding to the segment KM) from the point K to the reception element 12 is different from the distance (corresponding to the segment KN) from the point K to the reception element 16. Since the distance of the segment KN is larger than the distance of the segment KM, the acoustic pressure of acoustic waves originating from the point K received by the reception element 16 is smaller than the acoustic pressure of acoustic waves originating from the point K received by the reception element 12.

FIG. 4B is a diagram illustrating the light intensity on the segment EI. Moreover, FIG. 4C is a diagram illustrating the light intensity on the segment HF. In FIGS. 4B and 4C, a case in which the length of the segment EM is the same as the length of the segment HO, and the light absorbers present at the points E and H have the same size and absorption coefficient is considered. In this case, if the light quantities at the points E and H are different, the intensities of the acoustic waves generated from the light absorbers at the points E and H are different. That is, these acoustic waves have approximately the same frequency but have different initial acoustic pressure levels. Thus, the reception element 12 receives acoustic waves having approximately the same frequency as and different acoustic pressure from the acoustic waves received by the reception element 14. If the light absorbers present at the points E and H have different sizes, the acoustic waves generated at the respective points have different frequencies, and the reception elements 12 and 14 receive acoustic waves having different frequencies and acoustic pressure levels. If the frequencies of generated acoustic waves are different, the degrees of attenuation of the acoustic waves resulting from the frequency-dependent attenuation are different even if the acoustic waves have propagated the same distance.

FIG. 4D is a diagram illustrating the relation between the intensity of the light intensity on the segment GJ and the depth. That is, FIG. 4D illustrates a state in which the intensity of light intensity decreases exponentially from the point J located close to a light irradiation side toward the point G on the side distant from the irradiation side.

In FIGS. 4B, 4C, and 4D, the acoustic waves received by the reception elements 12, 14, and 16 are different based on the light distribution area 9 and the positions of the respective reception elements 12, 14, and 16. The reason why the positions of the reception elements 12, 14, and 16 affect the received acoustic waves is because the distances from an acoustic wave generation point (assumed to be the same point in the object 10) to the respective reception elements 12, 14, and 16 are different. That is, when the reception elements 12, 14, and 16 are disposed in a distributed manner as illustrated in FIG. 4A, the light intensity profile (light intensity distribution) on the normal line of each of the reception elements 12, 14, and 16 may differ remarkably depending on the positional relation between the light distribution area 9 and the reception elements 12, 14, and 16. Further, the degree of attenuation of acoustic waves reaching the reception elements 12, 14, and 16 disposed in a distributed manner may differ remarkably in respective reception elements 12, 14, and 16 even if the acoustic waves were generated at the same point in the object 10. Thus, when the reception elements 12, 14, and 16 are disposed in a distributed manner, it is necessary to optimize the level of correction for the attenuation of acoustic pressure of acoustic waves having predetermined frequency characteristics for the respective reception elements 12, 14, and 16 to acquire significant signals contributing to visualization. Further, it is necessary to correct a decrease or a variation in the initial acoustic pressure resulting from attenuation of light intensity in the object 10 (that is, to adjust the level of light intensity correction) by taking the difference in light intensity profile of the reception elements 12, 14, and 16 into consideration.

FIG. 2C is a cross-sectional view illustrating another state in which photoacoustic waves are detected by a plurality of reception elements. A case in which the reception elements 12, 14, and 16 are present away from the object 10 as in FIG. 2C and a medium having very small frequency-dependent attenuation like water is filled between the reception elements 12, 14, and 16 and the object 10 will be considered. In this case, the influence of attenuation of acoustic waves can be ignored among the distance d1, d2, and d3 and only the attenuation of acoustic waves inside the object 10 may be taken into consideration. When the light irradiation time is set to t=0, the time at which acoustic waves reach the reception elements 12, 14, and 16 from the surface (in FIG. 2C, corresponding to points M, N, and O) of the object 10 is different for the respective reception elements 12, 14, and 16. After that, even if acoustic waves are received at the same time t=τ, when the distances between the reception elements 12, 14, and 16 and the object 10 are different, the occurrence depths of the acoustic waves in the object 10 when seen from the reception elements 12, 14, and 16 are different for the respective reception elements 12, 14, and 16. Thus, even if acoustic waves are received at the same time t=τ, the degrees of attenuation of the acoustic waves in the object 10 are different for the respective reception elements 12, 14, and 16. That is, when the reception elements 12, 14, and 16 are disposed in the above-described manner, the relationship between the acoustic wave reception time and the degree of attenuation of acoustic waves in the object 10 is different for the respective reception elements 12, 14, and 16. The level of correction for attenuation and the light intensity needs to be adjusted for the respective reception elements 12, 14, and 16 by taking the difference in the relationship into consideration.

### Example 1

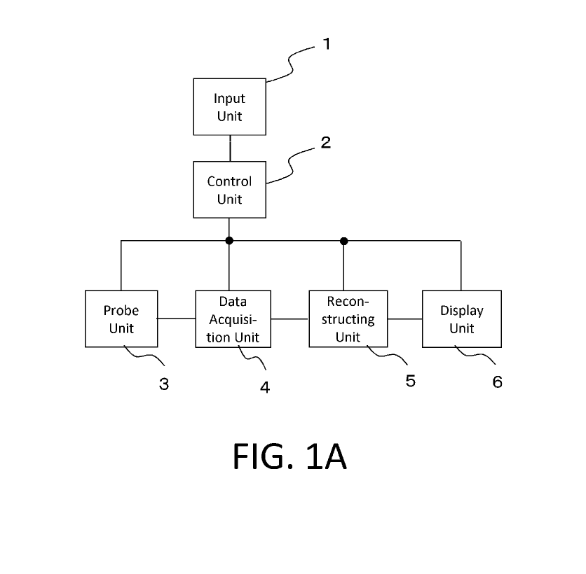

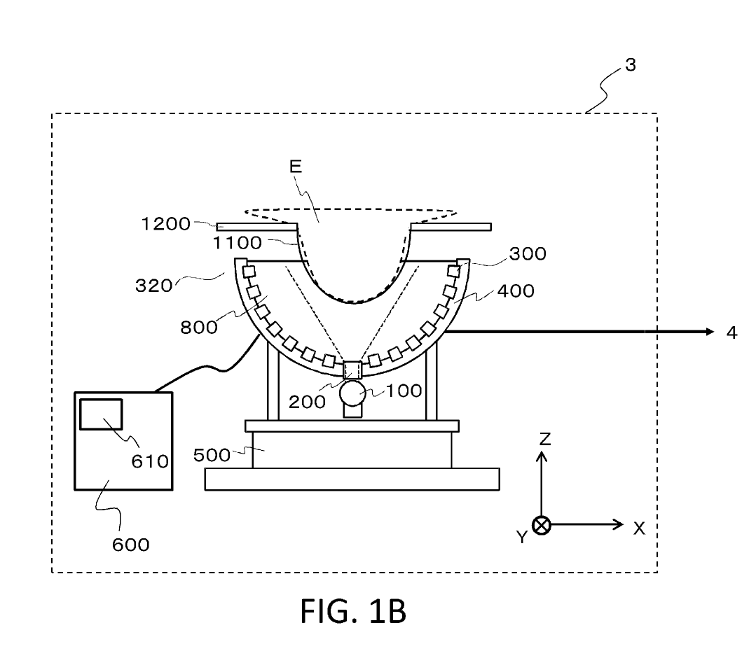

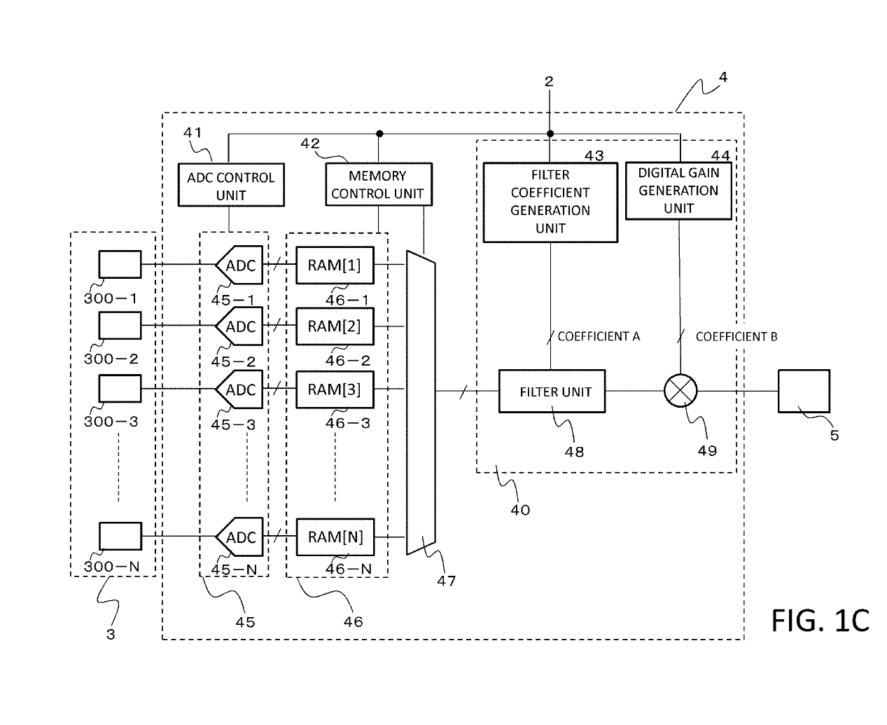

FIG. 1A is a block diagram illustrating Example 1 of an object information acquiring apparatus of the present invention. An object information acquiring apparatus 1000 (hereinafter referred to simply as an “apparatus 1000”) of Example 1 includes an input unit 1, a control unit 2, a probe unit 3, a data acquisition unit 4, a reconstructing unit 5, and a display unit 6 as its basic components. Hereinafter, the respective blocks will be described.

<<Apparatus Configuration>>

(Input Unit)

The input unit 1 includes an interface that receives the input of an optical characteristic value (an absorption coefficient or an equivalent scattering coefficient) of an object E, a value of the frequency dependent attenuation of the acoustic wave, and control information necessary for controlling the apparatus 1000 from an operator.

For example, when the object E is a living body, the input unit 1 may acquire the absorption coefficient, the equivalent scattering coefficient, and a value of the frequency dependent attenuation directly. Also, the input unit 1 may acquire known statistical values corresponding to the characteristics (e.g. age) of the object E.

Also, when the age or the gender is input, the values corresponding thereto (e.g. the optical characteristic value and the value of the frequency dependent attenuation of the acoustic wave) may be automatically set.

Alternatively, values measured by another apparatus may be input. The input unit 1 outputs the input optical characteristic value of the object E and the control information to the control unit 2 as setting values.

Also, the input unit 1 may acquire a target position or target region to acquire the characteristic information of the object.

Furthermore, the data acquisition unit 4 may determine a frequency filter (described below) based on the information acquired by the input unit 1 (e.g. target position).

The input unit 1 is configured such that users can designate desired information to input desired information to the control unit 2. Examples of the input unit 1 include a keyboard, a mouse, a touch panel, a dial, a button, and the like. When a touch panel is used as the input unit 1, the touch panel may be configured such that the display unit 6 also serves as the input unit 1.

(Control Unit)

The control unit 2 performs a process of supplying various parameters input from the input unit 1 to other blocks at appropriate timings or supplying image reconstruction control information of the apparatus 1000 to other blocks to control the entire apparatus 1000. The control unit 2 is typically configured as a PC or an electronic board on which a circuit such as a CPU, a FPGA, or an ASIC is mounted. However, the constituent elements of the control unit 2 are not necessarily limited to these elements. An arbitrary constituent element may be used as long as the apparatus 1000 can be controlled.

(Probe Unit)

The probe unit 3 irradiates the object E with light, receives acoustic waves generated inside the object E, converts the acoustic waves to electrical signals, and transmits the electrical signals to the data acquisition unit 4.

(Data Acquisition Unit)

The data acquisition unit 4 is a functional block that performs a process of receiving the electrical signals from the probe unit 3 and the signals from the control unit to generate digital signals corresponding to one or more reception elements. The data acquisition unit 4 outputs the digital signals generated in this manner to the reconstructing unit 5 on the subsequent stage.

(Reconstructing Unit)

The reconstructing unit 5 generates (reconstructs) image data of one or more constituent points inside the object E using the digital signals input from the data acquisition unit 4 and supplies the image data to the display unit 6 on the subsequent stage. A time-domain or Fourier-domain back-projection method which is generally used in the tomography technique can be used as the reconstruction method. The image data of the present example may be 2-dimensional or 3-dimensional data indicating information (living body information such as an initial acoustic pressure distribution, an absorption coefficient distribution, or an oxygen saturation in the living body) on image reconstruction points inside the object E.

(Display Unit)

The display unit 6 displays a photoacoustic image inside the object E and numerical data of a specific region of interest (ROI) to the operator using the image data input from the reconstructing unit 5. The photoacoustic image is an image obtained by arranging items of information on the constituent points inside the object E in one dimension, two dimensions, or three dimensions. A liquid crystal display or the like is typically used as the display unit 6. However, the display unit is not limited thereto, but other types of display such as a plasma display, an organic EL display, or a FED may be used. The display unit 6 may be provided as a separate member from the apparatus 1000.

FIG. 1B is a schematic diagram illustrating the configuration of a probe unit 3 according to Example 1, and the portions corresponding to those in FIGS. 1A and 1B will be denoted by the same reference numerals and the description thereof will be omitted unless necessary. The probe unit 3 includes a light source 100, an optical system 200, a plurality of reception elements 300, a support 400, a scanner 500 as a moving mechanism, an acoustic matching material 800, a shape holding portion 1100, an attachment portion 1200 as its basic components. Hereinafter, the configuration of the probe unit 3 will be described in detail.

<<Configuration of Probe Unit>>

(Object)

The object E is not a constituent element of the probe unit 3, but will be described herein because the object E is the subject measured by the probe unit 3. An example of the object E includes a living body such as a breast and a phantom that simulates the acoustic characteristics and the optical characteristics of a living body and that is specialized for use in adjustment of the apparatus 1000. The acoustic characteristics specifically mean the propagation velocity and the attenuation factor of acoustic waves, and the optical characteristics specifically mean the absorption coefficient and the scattering coefficient of light. A light absorber having a large absorption coefficient is present inside the object E, and examples of such a light absorber in a living body include hemoglobin, water, melanin, collagen, and fat. In a phantom, a substance that simulates the optical characteristics may be enclosed in the object as the light absorber. The object E is depicted by a dot line in FIG. 1B for the sake of convenience.

(Light Source)

The light source 100 generates a pulsed beam. The light source 100 is preferably a laser in order to obtain a large output but may be a light emitting diode or the like and has to emit light in a sufficient short period according to the thermal characteristics of the object E in order to generate photoacoustic waves effectively. The pulse width of a pulsed beam which is the light generated from the light source 100 is preferably several tens of nano seconds or smaller when the object is a living body. The wavelength of the pulsed beam is in a near-infrared region called the window of a living body and is preferably between approximately 700 nm and 1200 nm. Since light in this wavelength region can reach up to a relatively deeper portion of a living body, it is possible to obtain the information on the deep portion of the living body. The wavelength of the pulsed beam may be approximately between 500 nm and 700 nm which ranges from the visible light region to the near-infrared region if the purpose is limited to measurement of the surface portion of a living body. Further, the wavelength of the pulsed beam preferably has a higher absorption coefficient than a measurement target.

(Optical System)

The optical system 200 guides a pulsed beam generated by the light source 100 to the object E, and examples thereof include an optical device such as a lens, a mirror, a prism, an optical fiber, or a diffuser. The optical system 200 may appropriately change the shape or the optical density of light so as to have a desired light distribution using these optical devices when guiding light. However, the present invention is not limited thereto, but the optical device may be an arbitrary device as long as the device performs the above-described function. In the present example, the optical system 200 is configured to irradiate light about the central region of curvature of a hemisphere.

Moreover, as for the intensity of light permitted to irradiate a living body tissue, maximum permissible exposure (MPE) is defined by the following safety standards. For example, IEC 60825-1: Safety of laser products. Alternatively, JIS C 6802: Safety standards for laser products. Alternatively, FDA: 21CFR Part 1040.10. Alternatively, ANSI 2136.1: Laser Safety Standards. The maximum permissible exposure defines the intensity of light that can be irradiated per unit area. Thus, an optical system can guide a larger amount of light toward the object E by collectively irradiating a large area of the surface of the object E with light. Thus, the probe unit 3 can receive photoacoustic waves with high SN ratio. Therefore, it is preferable to condense the light from the light source 100 using a lens to that the light broadens to a certain area as indicated by a broken line in FIG. 1B.

(Reception Element)

The reception element 300 is an element that receives photoacoustic wave to convert the same to electrical signals. The photoacoustic waves from the object E preferably have high reception sensitivity and a wide frequency range. The reception element 300 is configured as a piezoelectric ceramic material represented by PZT (lead zirconate titanate) or a polymer piezoelectric film material represented by PVDF (polyvinylidene fluoride resin). However, the present invention is not limited thereto but an element other than piezoelectric elements may be used. For example, an electrocapacitance element such as cMUT (Capacitive micromachined ultrasonic transducers) or a reception element which uses a Fabry-Perot interferometer may be used.

(Support)

The support 400 is an approximately hemispherical container in which a plurality of reception elements 300 is provided on the inner surface of the hemisphere and the optical system 200 is provided at the bottom (pole) of the hemisphere. Moreover, the acoustic matching material 800 described later is filled inside the hemisphere of the support 400. The support 400 is preferably configured using a metal material having high mechanical strength in order to support these members. In the support 400, the plurality of reception elements 300 is disposed along the hemispherical shape on the inner side of the support 400 and the reception directions of the respective reception elements 300 are different. The reception elements 300 are disposed in an array so as to face the center of curvature of the hemispherical shape of the support 400. That is, the plurality of reception elements 300 are disposed in the support 400 so that photoacoustic waves generated at the center of curvature of the hemispherical shape of the support 400 and the areas near the center of curvature can be received particularly with high sensitivity. The receiving unit 320 includes the reception element 300 and the support 400 as its basic components.

(Scanner)

The scanner 500 moves the support 400 in X, Y, and Z-directions. The scanner 500 is a moving mechanism that changes the position of the support 400 in relation to the object E by doing so. The scanner 500 includes guide mechanisms for X, Y, and Z-directions (not illustrated), driving mechanisms for X, Y, and Z-directions, and position sensors that detect the positions in the X, Y, and Z-directions of the support 400. Since the support 400 is loaded above the scanner 500, a linear guide or the like that can endure large load may be preferably used as the guide mechanism. Moreover, a lead screw mechanism, a link mechanism, a gear mechanism, a hydraulic mechanism, and the like can be used as the driving mechanism. The driving mechanism may supply the driving force thereof from a motor or the like. A potentiometer or the like that uses an encoder, a variable resistor, or the like can be used as the position sensor, for example. However, the present invention is not limited thereto, and the position of the object E may be changed in relation to the support 400 and alternatively, both the object E and the support 400 may be moved. That is, it is sufficient that the positional relation between the object E and the support 400 is changed. Moreover, the scanner 500 may preferably realize the movement continuously to change the positional relation but may move in a stepwise manner. Although the scanner 500 is preferably an electric stage, the scanner 500 may be a manual stage. However, the present invention is not limited thereto, but an arbitrary member may be used as long as the member is configured to move at least one of the object E and the support 400.

(Image Acquiring Unit)

An image acquiring unit 600 includes a camera and an imaging device capable of imaging the object E such as a transducer that transceives acoustic waves, for example. A transducer provided separately from the plurality of acoustic wave reception elements 300 and at least one elements of the plurality of acoustic wave reception elements 300 can be employed as the transducer. The transducer is provided so as to be able to transmit acoustic waves and receive reflection waves of the acoustic waves. A captured image processing unit 610 present inside the image acquiring unit 600 may acquire a captured image based on the reception signals output from the imaging device and may acquire shape information (corresponding to a presence range of the object) of the object E by performing image processing based on the captured image. Moreover, the captured image processing unit 610 may acquire the shape information of the object E using a three-dimensional measurement technique such as a stereo method based on the captured images obtained from a plurality of directions. In this case, the imaging device and the captured image processing unit may be collectively referred to as a shape acquiring unit 600. The shape acquiring unit 600 may be provided as a separate member from the apparatus 1000.

(Acoustic Matching Material)

The acoustic matching material 800 is configured to fill the space between the object E and the reception element 300 to acoustically couple the object E and the reception element 300. The acoustic matching material 800 may be provided between the reception element 300 and the shape holding portion 1100 and between the shape holding portion 1100 and the object E. Moreover, the acoustic matching material 800 disposed between the reception element 300 and the shape holding portion 1100 may be different from the acoustic matching material 800 disposed between the shape holding portion 1100 and the object E. The acoustic matching material 800 is preferably formed of a material of which the acoustic impedance is close to that of the object E and the reception element 300. Further, the acoustic matching material 800 is more preferably formed of a material of which the acoustic impedance is between those of the object E and the reception element 300. Moreover, the acoustic matching material 800 is preferably formed of a material that transmits pulsed beams generated by the light source 100. The acoustic matching material 800 is preferably liquid. Examples of the acoustic matching material 800 include water, castor oil, gel, and the like.

The probe unit 3 includes the respective constituent elements described above.

FIG. 1C is a block diagram illustrating an inner configuration of the data acquisition unit 4 according to Example 1, and the portions corresponding to those in FIG. 1A or 1B will be denoted by the same reference numerals and the description thereof will be omitted unless necessary. The data acquisition unit 4 includes an analog-to-digital converter (ADC) control unit 41, an ADC unit 45, a memory control unit 42, a memory unit 46, a selector 47, and a digital gain unit 40 as its basic components. The digital gain unit 40 (corresponding to a correction unit) includes a filter coefficient generation unit 43, a filter unit 48 (corresponding to an extraction processing unit), a digital gain generation unit 44, and a multiplication unit 49.

(ADC Control Unit)

The ADC control unit 41 supplies an operating voltage, sampling clocks, a sampling start command signal, an ADC setting parameter, and the like to the ADC unit 45 (corresponding to a conversion unit).

(ADC Unit)

The ADC unit 45 samples the electrical signals from the reception element 300 at a predetermined frequency based on the sampling clocks output by the ADC control unit 41 based on the information supplied from the ADC control unit 41. The ADC unit 45 converts (corresponding to analog-to-digital conversion) the electrical signals obtained by the sampling to digital signals and outputs the digital signals to the memory unit 46. In the ADC unit 45, a total number of ADCs 45-1 to 45-N is not necessary the same as the total number of reception elements 300. In the ADC unit 45, when the total number of reception elements 300 is larger than the total number of ADCs 45-1 to 45-N, an analog switch (not illustrated) may be provided between the reception element 300 and the ADC 45-k (k=1, 2, N). The analog switch may be configured to be able to change a mode in which the reception element 300 and the ADC 45-k (k=1, 2, . . . , N) are connected. By doing so, the ADC unit 45 may sample the acoustic waves received by the plurality of reception elements 300 using one ADC 45-k. A case in which the total number of reception elements 300 is smaller than the total number of ADCs 45-1 to 45-N will be considered. In this case, an analog switch may be provided between the reception element 300 and the ADC 45-k, and acoustic waves received by one reception element 300 may be connected to a plurality of ADCs 45 and may be sampled. As described above, the ADC unit 45 appropriately determines the total number of ADCs 45-k and a mode in which the analog switch connects the ADCs 45-k and the reception elements 300 by taking various restrictions of the entire apparatus 1000, the total number of reception elements 300, and the like into consideration.

(Memory Control Unit)

The memory control unit 42 supplies an operating voltage, write clocks, read clocks, a write enable, a read enable, a write address, a read address, and the like to the memory unit 46. Moreover, the memory control unit 42 supplies memory selection information to the selector 47.

(Memory Unit)

The memory unit 46 includes memories that store digital signals input from the ADC unit 45 based on the write clocks and the write enable output by the memory control unit 42.

(Selector)

The selector 47 selects one memory 46-k (RAM[k]) (k=1, 2, . . . , N) from the memories 46-1 to 46-N of the memory unit 46 based on an instruction signal input from the memory control unit 42. Moreover, the selector 47 connects the selected memory 46-k to the digital gain unit 40. Further, the selector 47 reads digital signals stored in the selected memory 46-k based on the read clocks, the read enable, the read address, and the like supplied from the memory control unit 42 and outputs the digital signals to the digital gain unit 40.

(Digital Gain Unit)

The digital gain unit 40 includes the filter coefficient generation unit 43, the digital gain generation unit 44, the filter unit 48, and the multiplication unit 49 as its basic components. The digital gain unit 40 performs a filtering process and a gain process on the digital signals output by the memory selected by the selector 47.

The filter coefficient generation unit 43 receives a control signal supplied from the control unit 2, generates a filter coefficient based on the control signal, and supplies the filter coefficient to the filter unit 48.

The filter unit 48 performs a filtering process on the digital signals output by the memory 46-k (k=1, 2, . . . , N) selected by the selector 47 using the filter coefficient input from the filter coefficient generation unit 43. The filter unit 48 sets a pass-band (corresponding to a predetermined frequency range) corresponding to the filter coefficient supplied by the filter coefficient generation unit 43. The filter unit 48 is preferably formed of a FIR filter but is not necessarily limited thereto. The configuration of the filter unit 48 can be appropriately changed as long as the filter unit can substantially realize a convolution operation between an impulse response and the digital signals output by the memory 46-k. Moreover, the configuration of the filter coefficient generation unit 43 can be appropriately changed similarly to the above. Such an operation may be an operation in a frequency domain and an operation in a time domain, and the other operations may be used. Moreover, the filter unit 48 may be a band-pass filter, a high-pass filter, a low-pass filter, or a combination thereof and is not limited to a specific format as long as the filter unit can extract a desired frequency range. In the present example, a process of extracting a desired frequency range will be referred to as a filtering process. For example, the filtering process is such a process of extracting the predetermined frequency range corresponding to a filter coefficient by cutting off frequency components outside the predetermined frequency range.

The digital gain generation unit 44 generates a gain value based on the control information supplied from the control unit 2 and supplies the gain value to the multiplication unit 49.

The multiplication unit 49 multiplies the filtered digital signal output by the filter unit 48 by the gain value supplied by the digital gain generation unit 44. Moreover, the multiplication unit 49 supplies the multiplied digital signal (that is, the digital signal having been subjected to the filtering process and the gain process) to the reconstructing unit 5. For example, the filtering process is a process of extracting a predetermined frequency range corresponding to the filter coefficient by cutting off frequency components outside the predetermined frequency range.

In the present example, one digital gain unit 40 illustrated in FIG. 1C is shared by N memories. However, the present invention is not limited thereto but one digital gain unit 40 may be disposed for one memory. Moreover, a configuration in which one digital gain unit 40 is shared by a plurality of memories (smaller than N) and a plurality of digital gain units 40 are present may be employed.

<<Description of Operation of Object Information Acquiring Apparatus>>

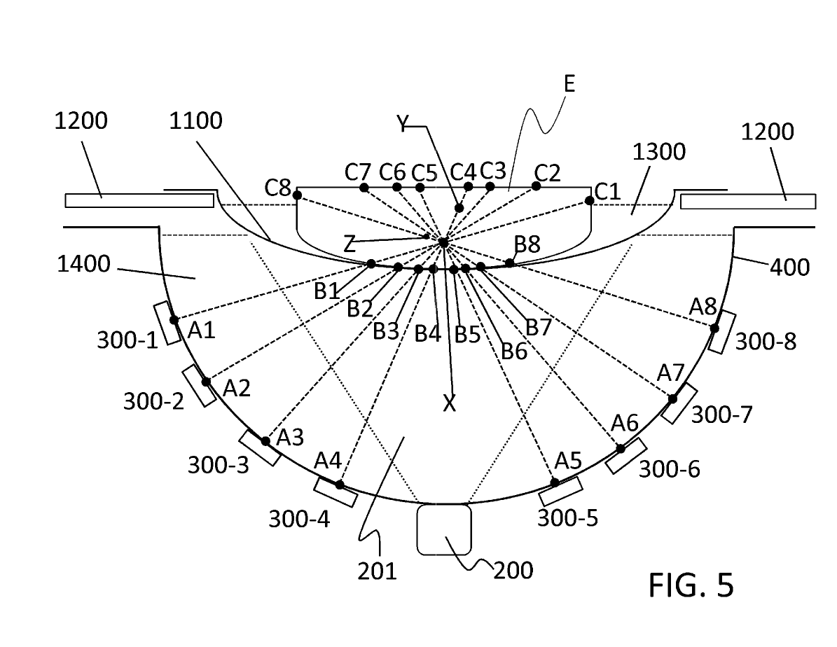

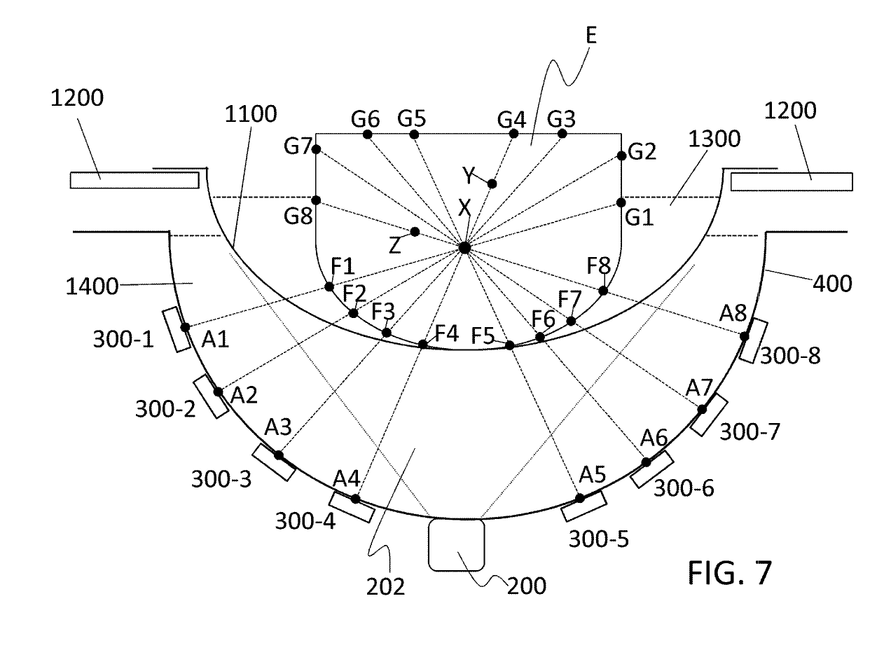

FIGS. 5 and 7 illustrate the state during photoacoustic measurement of the apparatus 1000, and the size of the object E in FIG. 5 is different from that of FIG. 7. Thus, the distance from the reception elements 300-1 to 300-8 to the object E in FIG. 5 is different from that of FIG. 7. That is, the positional relation between the object E and the reception elements 300-1 to 300-8 in FIG. 5 is different from that of FIG. 7. Moreover, in the present example, it is assumed that the positional relation between the object E and the support 400 illustrated in FIGS. 5 and 7 is fixed.

In FIG. 5, first, the object E is irradiated with light from the optical system 200, and the acoustic waves generated inside the object E are received by the reception elements 300-1 to 300-8. For example, the reception element 300-4 receives acoustic waves generated from a point on a segment B4-C4 with high sensitivity.

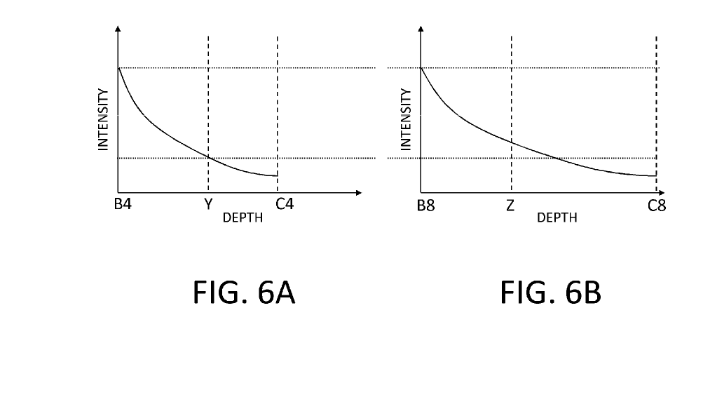

FIGS. 6A and 6B are diagrams illustrating the relation between the light intensity and the depth during photoacoustic measurement according to Example 1. FIG. 6A is a diagram illustrating the relation between the light intensity and the depth on a segment B4-C4 in FIG. 5. That is, it is assumed that the light intensity in the object E is not uniform and the light intensity on the segment B4-C4 is in such a state as illustrated in FIG. 6A.

FIG. 6B is a diagram illustrating the relation between the light intensity and the depth on a segment B8-C8. It is assumed that the light intensity on the segment B8-C8 is in such a state as illustrated in FIG. 6B. The reception element 300-8 receives acoustic waves generated from a point on the segment B8-C8 with high sensitivity. Here, it is assumed that a segment An-Cn is present on a normal line of a point An on the reception element 300-n. Moreover, it is assumed that since a segment An-Bn (n=1 to 8) is filled with a medium of which the frequency-dependent attenuation is very smaller than that of the object E, the influence of attenuation can be ignored. Even if the length of a segment B4-Y is the same as the length of a segment B8-Z and the light absorbers present at the points Y and Z have the same size and the same absorption coefficient, since the light intensities at the points Y and Z are different as illustrated in FIGS. 6A and 6B, the initial acoustic pressure levels of the acoustic waves generated at the points Y and Z are different. That is, FIGS. 6A and 6B illustrate light intensity profiles in acoustic wave reception areas of the reception elements 300-4 and 300-8 in FIG. 5. When the reception element 300-4 receives acoustic waves generated in the segment B4-C4, the light intensity profile on the segment B4-C4 is as illustrated in FIG. 6A. Moreover, when the reception element 300-8 receives acoustic waves generated in the segment B8-C8, the light intensity profile on the segment B8-C8 is as illustrated in FIG. 6B. As can be understood from comparison between FIGS. 6A and 6B, the generation areas of the acoustic waves that the reception elements 300-4 and 300-8 receive have different light intensity profiles.

In FIG. 7, the reception element 300-4 receives acoustic waves generated from a point on a segment F4-G4 with high sensitivity. Here, the data acquisition unit 4 performs the following sampling on electrical signals output when the reception elements 300-1 to 300-8 receive the acoustic waves which have reached the reception elements 300-1 to 300-8 and originate from the segment Bn-Cn illustrated in FIG. 5. That is, the ADCs 45-1 to 45-8 in the data acquisition unit 4 perform sampling on the electrical signals at a predetermined sampling cycle. Alternatively, in the data acquisition unit 4, the electrical signals output when the reception elements 300-1 to 300-8 receive the acoustic waves originating from a segment Fn-Gn illustrated in FIG. 7 are sampled by the ADCs 45-1 to 45-8 at a predetermined sampling cycle. Moreover, the data acquisition unit 4 converts the sampled electrical signals to digital signals and outputs the digital signals to the memories 46-1 to 46-8. The memories 46-1 to 46-8 stores the digital signals output by the ADCs 45-1 to 45-8 based on the write clocks, the write enable, the write address, and the like output by the memory control unit 42. For example, the cycle of the write clocks that define the timings to write data to the memories 46-1 to 46-8 is the same as the sampling cycle of the ADCs 45-1 to 45-8. In the memory control unit 42, sampling of acoustic waves starts based on a light irradiation time and the write clocks are supplied to the memory unit 46 every write clock cycle from the start of sampling so that the memories 46-1 to 46-8 can sequentially write data thereto. That is, the memories 46-1 to 46-8 store the digital signals output by the ADCs 45-1 to 45-8 every clock cycle.

The cycle of the sampling clocks of the ADC 45 may not necessarily be the same as the cycle of the write clocks of the memory 46. For example, the frequency of the write clocks of the memory 46 is set to X times the frequency of the sampling clocks of the ADC 45 and the write enable of the memory 46 is enabled every X cycles of the write clocks of the memory 46. By doing so, a process of making the cycle of the sampling clocks of the ADC 45 substantially the same as the cycle of the write clocks of the memory 46 may be performed. Moreover, the relationship between the cycle of the sampling clocks of the ADC 45 and the cycle of the write clocks of the memory 46 is not necessarily limited thereto, but an arbitrary method may be used as long as the method can acquire digital signals necessary for image reconstruction.

When acquisition of the digital signals is completed, the memory control unit 42 controls the selector 47 to allow the selector 47 to select one of the memories 46-1 to 46-8 and receive the digital signals stored in the selected memory. The selector 47 outputs the received digital signals to the filter unit 48. The filter unit 48 extracts signals of a predetermined frequency range included in the digital signals input from the selector 47 and outputs the extracted signals to the multiplication unit 49. In this case, the filter coefficient generation unit 43 generates a filter coefficient for extracting a predetermined frequency range based on the control signal from the control unit 2 and outputs the filter coefficient to the filter unit 48. The filter unit 48 receives the filter coefficient and determines the predetermined frequency range to be extracted based on the filter coefficient.

The multiplication unit 49 multiplies the digital signals from the filter unit 48 by a digital gain value for correcting the frequency-dependent attenuation of acoustic waves in the object E and the distance-dependent attenuation caused by energy dissipation due to spherical wave propagation or cylindrical wave propagation and supplies the multiplied digital signals to the reconstructing unit 5. The digital gain generation unit 44 generates a digital gain value based on the control content from the control unit 2 and supplies the digital gain value to the multiplication unit 49. The multiplication unit 49 outputs the digital signals multiplied (corrected) in the above-described manner to the reconstructing unit 5. The digital gain generation unit 44 may set the digital gain patterns used for attenuation correction to be supplied to the multiplication unit 49 so as to be different for the digital signals based on the respective acoustic waves received by the different reception elements 300-1 to 300-8.

In this example, the apparatus 1000 applies different frequency filters to the plurality of the digital signals stored in the memory in accordance with a positional relation between a position in which characteristic information of the object is acquired and a position of the acoustic wave detection elements. The frequency filter represents a gain of each frequency component of digital signal data.

The reconstructing unit 5 generates photoacoustic image data based on the digital signals input from the multiplication unit 49.

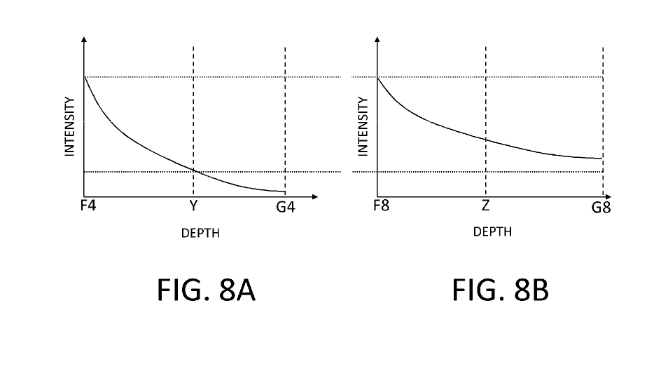

FIGS. 8A and 8B are diagrams illustrating the relation between the light intensity and the depth during another photoacoustic measurement according to Example 1. FIG. 8A is a diagram illustrating the relation between the light intensity and the depth on a segment F4-G4. That is, it is assumed that the light intensity in the object E is not uniform and the light intensity on the segment F4-G4 is in such a state as illustrated in FIG. 8A.

FIG. 8B is a diagram illustrating the relation between the light intensity and the depth on a segment F8-G8. That is, it is assumed that the light intensity in the object E is not uniform and the light intensity on the segment F8-G8 is in such a state as illustrated in FIG. 8B. Here, it is also assumed that the segment An-Gn is present on a normal line of a point An on the reception element 300-n. Moreover, since a segment An-Fn (n=1 to 8) is filled with a medium of which the frequency-dependent attenuation is very smaller than that of the object E, the influence of the frequency-dependent attenuation can be ignored.

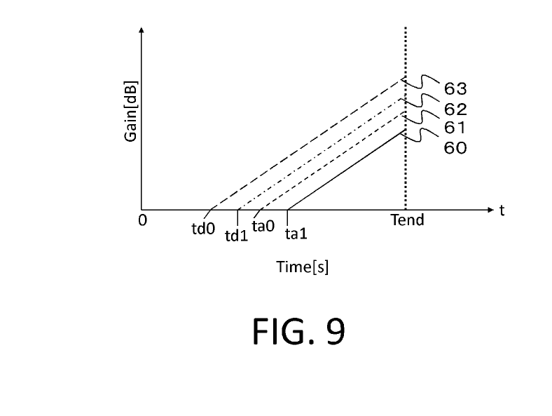

FIG. 9 is a diagram illustrating the relation between the digital gain and the time according to Example 1. That is, FIG. 9 illustrates a case in which sampling starts at the light irradiation time t=0. FIG. 9 illustrates the correspondence in such a case between the sampling time of digital signals in the ADCs 45-1 to 45-8 and the digital gain pattern multiplied by the multiplication unit 49 for the digital signals sampled at a certain time. In this case, for example, the filter unit 48 extracts signals having the central frequency of 1 MHz. Here, a digital gain pattern 60 indicates a digital gain pattern multiplied with the digital signals based on the acoustic waves received by the reception element 300-4 of FIG. 5. A digital gain pattern 61 indicates a digital gain pattern multiplied with the digital signals based on the acoustic waves received by the reception element 300-1 of FIG. 5. A digital gain pattern 62 indicates a digital gain pattern multiplied with the digital signals based on the acoustic waves received by the reception element 300-4 of FIG. 7. A digital gain pattern 63 indicates a digital gain pattern multiplied with the digital signals based on the acoustic waves received by the reception element 300-1 of FIG. 7. Here, it is assumed that time ta1 is the time at which, when the light irradiation time is t=0, acoustic waves generated at the point B4 propagate through the segment B4-A4 to reach the point A4. Moreover, it is assumed that time ta0 is the time at which acoustic waves generated at the point B1 propagate through the segment B1-A1 to reach the point A1. That is, ta0={(Length of Segment B1-A1)/(Acoustic velocity in media 1300 and 1400)} and ta1={(Length of Segment B4-A4)/(Acoustic velocity in media 1300 and 1400)}. In the present example, it is assumed that the acoustic velocity in the medium 1300 is the same as that of the medium 1400. When the acoustic velocities in the media 1300 and 1400 are different, the time ta0 and ta1 may be determined by taking the difference in the acoustic velocities into consideration.

FIG. 9 illustrates a case in which the multiplication unit 49 multiplies (corrects) the digital signals sampled after the acoustic waves generated on the surface of the object E reached the reception element 300 by the digital gain value. Since the digital gain value may have a value smaller than 1, the value of the digital signals obtained by being multiplied by the digital gain value may have a value smaller than the value before multiplication. The multiplication unit 49 multiplies a larger digital gain value as the sampling period of acoustic waves elapses in order to correct such attenuation of acoustic waves in the object E as illustrated in FIG. 3. That is, the digital gain generation unit 44 generates a larger digital gain value for attenuation correction since the acoustic waves reaching the surface of the object is attenuated more as the occurrence depth of the acoustic waves in the object E increases. The multiplication unit 49 may change the gain value to be multiplied according to the depth in the object for the respective reception elements 300. Moreover, the digital gain unit 40 performs a process of extracting signals in a desired frequency range only with the aid of the filter unit 48. However, the present invention is not limited thereto but the filter unit 48 may not be provided and the multiplication unit 49 may cause the digital gain unit 40 to multiply 0 as a gain value for signals outside the desired frequency range. In this manner, substantially the same effect as the process of cutting off signal components outside the desired frequency range using a filter may be obtained.

In FIG. 5, since the length of the segment A1-B1 is smaller than the length of the segment A4-B4, a digital gain is applied to the digital signals of the acoustic waves received by the reception element 300-1 at an earlier time than the digital signals of the acoustic waves received by the reception element 300-4. This state is depicted by the digital gain patterns 60 and 61 in FIG. 9. On the other hand, in FIG. 7, since the length of the segment A1-F1 is smaller than the length of the segment A4-F4, a digital gain is applied to the digital signals of acoustic waves received by the reception element 300-1 at an earlier time than the digital gains of the acoustic waves received by the reception element 300-4. This state is depicted by the digital gain patterns 62 and 63 in FIG. 9. Moreover, the lengths of the segment A1-B1 and the segment A4-B4 illustrated in FIG. 5 are larger than the lengths of the segment A1-F1 and the segment A4-F4 illustrated in FIG. 7. Thus, the time points td0 and td1 at which digital gains are applied in the state of FIG. 7 are earlier than the time points ta0 and ta1 at which digital gains are applied in the state of FIG. 5.

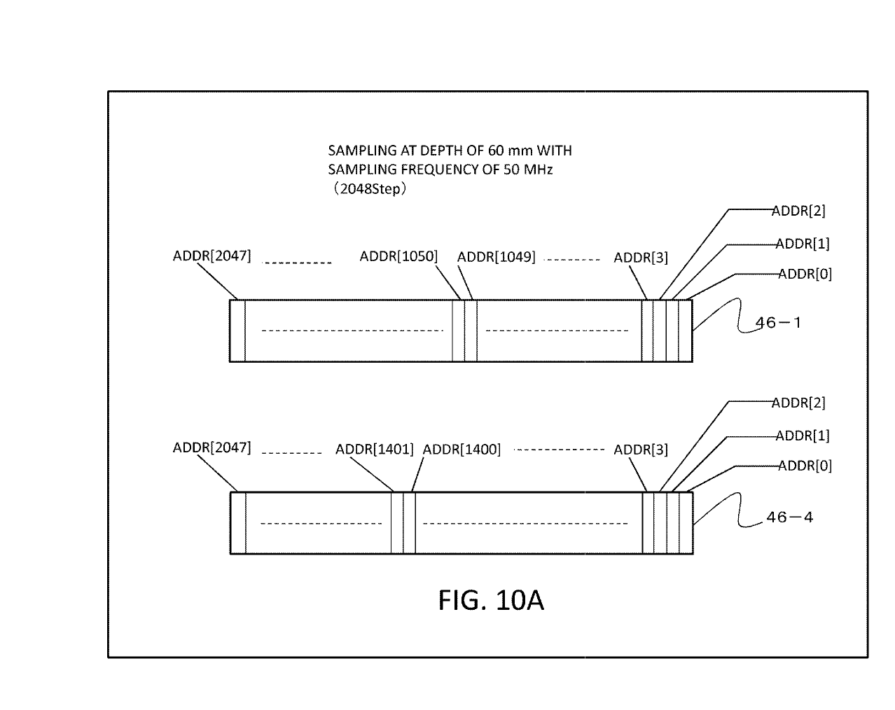

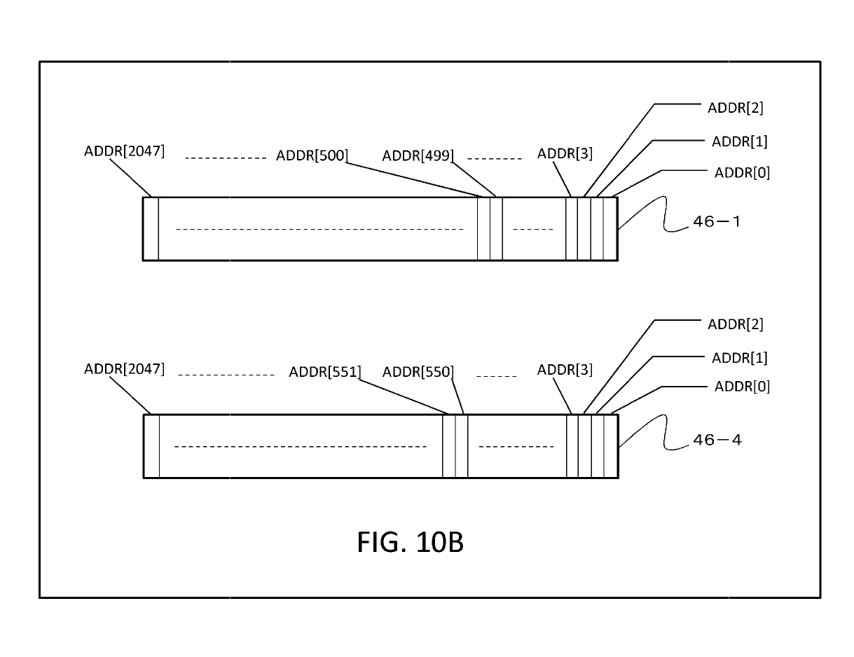

FIGS. 10A and 10B are diagrams illustrating a data storage state in a memory according to Example 1. FIG. 10A illustrates a data storage state when an object is irradiated with light at time t=0 and the memories 46-1 and 46-4 start storing the digital signals based on the irradiation in the state of FIG. 5. The memory 46-1 has a data storage area having the size of data area ADDR [2047:0]. In a partial data area ADDR [1049:0] of the entire data area ADDR [2047:0] of the memory 46-1, it is assumed that acoustic waves generated on the surface of the object E have not reached the reception element 300-1 and the digital signals thereof do not contain information on significant acoustic waves. In another partial data area ADDR [2047:1050] of the entire data area ADDR [2047:0] of the memory 46-1, digital signals sampled after the acoustic waves generated on the surface of the object E reach the reception element 300-1 are stored.

The multiplication unit 49 multiplies the digital signals stored in the data area ADDR [2047:1050] by a value corresponding to a time point later than the time point ta0 of the digital gain pattern 61. That is, the multiplication unit 49 performs the following process on the respective digital signals stored in the data area ADDR [2047:1050]. That is, the respective digital signals are multiplied by a digital gain value for correcting the frequency-dependent attenuation of acoustic waves in the object E and the distance-dependent attenuation caused by energy dissipation due to spherical wave propagation or cylindrical wave propagation. For example, the multiplication unit 49 multiplies a signal having a larger address number by a larger digital gain value like the digital gain pattern 61 of FIG. 9. As described above, the digital signals stored in the data area ADDR [1400:0] of the memory 46-4 of FIG. 10A do not contain information of significant acoustic waves. On the other hand, the data area ADDR [2047:1401] of the memory 46-4 stores digital signals sampled after the acoustic waves generated on the surface of the object E reach the reception element 300-1. Thus, the multiplication unit 49 multiplies the digital signals stored in the data area ADDR [2047:1401] by a value corresponding to a time point later than the time point ta1 of the digital gain pattern 60 illustrated in FIG. 9.

FIG. 10B is a diagram illustrating a data storage state of the memories 46-1 and 46-4 in the state of FIG. 7 when the light irradiation time is t=0 and storage of digital signals starts from t=0. In this example, similarly to the case of FIG. 10A, the digital signals stored in the data area ADDR [499:0] of the memory 46-1 and the digital signals stored in the data area ADDR [550:0] of the memory 46-4 do not contain information on significant acoustic waves. On the other hand, digital signals sampled after a predetermined time point are stored in the data area ADDR [2047:500] of the memory 46-1 and the data area ADDR [2047:551] of the memory 46-4. These digital signals sampled after the predetermined time point are digital signals which are sampled after the time at which the acoustic waves generated on the surface of the object E reach the reception elements 300-1 and 300-4. In this case, the multiplication unit 49 multiplies the digital data stored in the data area ADDR [2047:500] of the memory 46-1 by a value corresponding to a time point later than the time point td0 of the digital gain value 63. Moreover, the multiplication unit 49 multiplies the digital data stored in the data area ADDR [2047:550] of the memory 46-4 by a value corresponding to a time point later than the time point td1 of the digital gain value 62. That is, in the present example, a digital gain pattern (that is, attenuation correction mode) of acquired acoustic waves is changed according to the distance (that is, a positional relation) between the object E and the reception element 300.

FIG. 9 illustrates a digital gain pattern when the filter unit 48 extracts signals of which the control information is 1 MHz. However, the digital gain pattern may be changed according to a central frequency if the signals to be extracted have different central frequencies.

As described above, in photoacoustic imaging, the frequency range of acoustic waves generated inside the object E is several tens of MHz. It is not desirable to apply a uniform attenuation correction gain to signals having a wide frequency range. This is because the frequency-dependent attenuation is attenuation that depends on a frequency, and the degree of attenuation changes according to the frequency of acoustic waves even if the acoustic waves propagated the same distance in the object E. For example, the degree of attenuation in an object, of acoustic waves having the frequency of 1 MHz is approximately 10 times the degree of attenuation in an object, of acoustic waves having the frequency of 10 MHz. Thus, if a digital gain value ideal for the 1-MHz signal is multiplied for the 1-MHz and 10-MHz signals, a sufficient gain value is not secured for the 10-MHz signal. Thus, information corresponding to the 10-MHz signal may not be displayed sufficiently on a photoacoustic image, which may have an adverse effect on image diagnosis. Thus, in the present example, the filter unit 48 may extract signals in a predetermined frequency range from received acoustic waves using a filter, and the multiplication unit 49 may multiply an attenuation correction digital gain ideal for the frequency range. As described above, the higher the frequency of acoustic waves, the higher the degree of attenuation in the object. Thus, for example, the gain value for the 10-MHz signal may be set to be larger than the gain value for the 1-MHz signal. Moreover, a higher gain value may be applied such that the higher the predetermined frequency range extracted by the filter, the higher the applied gain value. Further, the filter unit 48 may extract a plurality of different frequency ranges and the multiplication unit 49 may multiply different digital gain values to the respective frequency ranges. In this way, an appropriate digital gain can be multiplied for signals having a wide frequency range. By doing so, acoustic waves in a wide frequency range can be visualized satisfactorily.

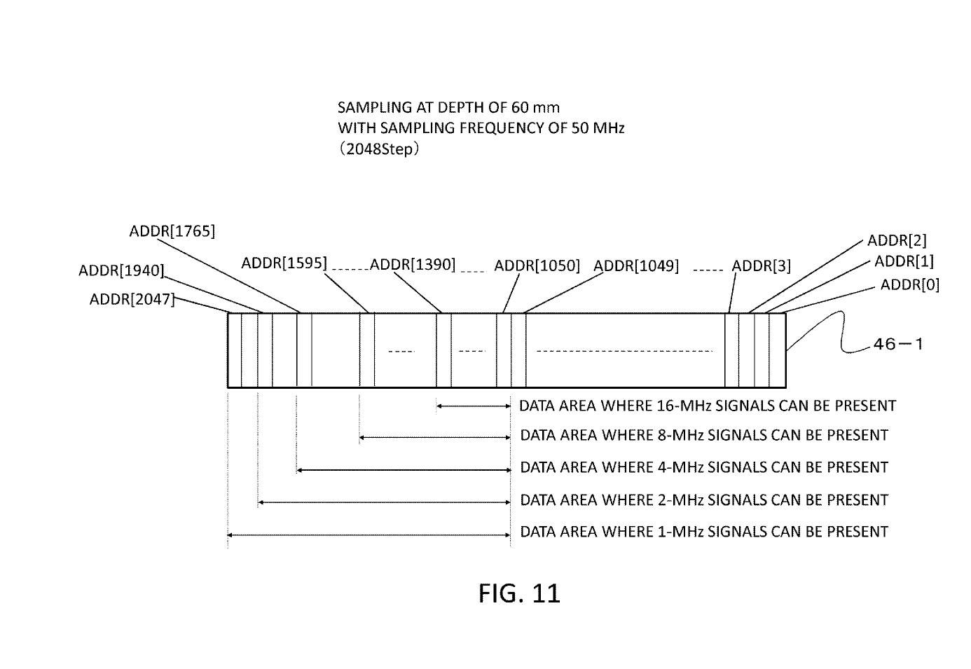

FIG. 11 is a diagram illustrating an acquisition state of digital signals in the memory 46-1 when acoustic waves received by the reception element 300-1 are sampled in the state illustrated in FIG. 5. This acquisition state is the same data acquisition state as the memory 46-1 illustrated in FIG. 10A. Digital signals of a portion corresponding to the segment A1-B1 filled with the media 1300 and 1400 are stored in the data area ADDR [1049:0], and these digital signals do not contain significant acoustic wave data. On the other hand, digital signals of a portion corresponding to the segment B1-C1 inside the object E are stored in the data area ADDR [2047:1050] and contain valid acoustic wave data.

In FIG. 3, it has been described that, when the absorption coefficients μ_{α} of the light absorbers (tumors or the like) in the object E have the same certain value, it is possible to predict the depth at which photoacoustic waves can be detected by the reception element for each frequency. In the present example, a frequency range that can be detected by the reception element 300-1 may be predicted based on the length of the segment B1-C1 inside the object E, the light intensity profile on the segment B1-C1, and an assumed value of the absorption coefficient μ_{α} of the light absorber in the state of FIG. 5. A statistical value for the age of a patient to be examined, a typical value of a living body corresponding to the average of these statistical values, and the value acquired by an apparatus that measures the absorption coefficient, provided separately from the apparatus 1000 of the present example may be used as the absorption coefficient μ_{α} of the light absorber. Moreover, when an absorption coefficient distribution of the object E is estimated from reception signals, the estimated value may be used.

In the case of FIG. 11, it is predicted that by the same method as FIG. 3, a 16-MHz signal is included in the data area ADDR [1390:1050] and an 8-MHz signal is included in the data area ADDR [1595:1050]. Moreover, it is predicted that a 4-MHz signal is included in the data area ADDR [1765:1050], a 2-MHz signal is included in the data area ADDR [1940:1050], and 1-MHz signal is included in the data area ADDR [2047:1050]. In this case, the memory control unit 42 selects the memory 46-1 with the aid of the selector 47 and supplies the digital signals stored in the data area ADDR [(1390+k):1050] of the memory 46-1 to the filter unit 48. The filter unit 48 extracts signals having the central frequency of 16 MHz among the supplied digital signals and allows the signals to pass therethrough. That is, the central frequency of the pass-band of the filter unit 48 is set to 16 MHz. Here, “k” in the data area ADDR [(1390+k):1050] is surplus data necessary for filtering the data in the data area ADDR [1390:1050]. k may be a parameter that depends on the number of filter taps but is not necessarily limited thereto. The filter coefficient generation unit 43 supplies a filter coefficient to the filter unit 48 so that the central frequency of the pass-band of the filter unit 48 becomes 16 MHz. The filter unit supplies the digital signals filtered so that the central frequency of the pass-band is 16 MHz to the multiplication unit 49. The digital gain generation unit 44 generates a digital gain pattern corresponding to the signal having the central frequency of 16 MHz and supplies the digital gain pattern to the multiplication unit 49.

The multiplication unit 49 multiplies the digital signals filtered so that the central frequency of the pass-band is 16 MHz by the value of the digital gain pattern input from the digital gain generation unit 44 and supplies the multiplied digital signals to the reconstructing unit 5.

The selector 47 selects the memory 46-1 based on the control signal from the memory control unit 42, receives the digital signals stored in the data area ADDR [(1595+k):1050] of the memory 46-1, and outputs the digital signals to the filter unit 48. The filter unit 48 filters the digital signals so that the central frequency of the pass-band is 8 MHz and supplies the filtered signals to the multiplication unit 49. The multiplication unit 49 multiplies a digital gain pattern ideal for the signals having the central frequency of 8 MHz, supplied from the filter unit 48 and supplies the multiplied signals to the reconstructing unit 5. By repeating the same processes as above, the filter unit 48 finally extracts signal components having the central frequencies of 16 MHz, 8 MHz, 4 MHz, 2 MHz, and 1 MHz from the digital signals stored in the memory 46-1. Moreover, the multiplication unit 49 multiplies the signal from which the respective signal components are extracted by the digital gain pattern for attenuation correction ideal for the respective frequencies. The digital signals subjected to attenuation correction in this manner are used for generation of photoacoustic image data in the reconstructing unit 5.

In the apparatus 1000 of the present example, the digital gain unit 40 may correct a difference in the light intensity profiles (hereinafter, this correction is referred to as light intensity correction). In such a case, since the digital gain pattern for light intensity correction can change depending on a positional relation between the reception element and the light intensity distribution, the digital gain pattern may be corrected based on a change in the positional relation. Moreover, in the present example, since the light irradiation pattern can be varied, the light intensity distribution may change depending on the light irradiation pattern. Thus, in the present example, a gain for correcting a difference in light intensity profile for each reception element, derived from the positional relation between the reception element and the light intensity distribution may be included in the digital gain pattern for attenuation correction. By doing so, it is possible to correct the attenuation in the respective reception elements and to optimize the light intensity correction. The digital gain unit 40 may perform light intensity correction by adjusting the digital gain value and may perform light intensity correction on the image data obtained after image reconstruction using the light intensity distribution data. Either light intensity correction method may be selected according to a user's setting.

The frequency of acoustic waves generated inside the object E depends on the size of a light absorber. In general, the light absorbers in a living body may have various sizes and the frequency range of generated acoustic waves may be several tens of MHz. Moreover, signals having such a wide frequency range can be generated inside the object E at a depth range of several cm. Thus, it is not appropriate to perform the same attenuation correction on signals having a frequency range of several tens of MHz. In the apparatus 1000 of the present example, even when acoustic waves having a wide frequency range of several tens of MHz are received, it is possible to perform attenuation correction ideal for each frequency range. The apparatus 1000 changes the number of filters to be applied and the pass-band of the filter according to the measurement depth inside the object E by predicting the frequency range of acoustic waves detectable by each reception element. That is, for example, the filter unit 48 changes the type of filters to be applied in parallel to the digital signal data stored in the memory 46-k so as to correspond to the elapse of the data acquisition time. By doing so, it is possible to efficiently perform a filtering process on many frequency ranges while saving the efforts for an unnecessary filtering process.

For example, in FIG. 11, the number of filter types to be applied in parallel decreases as the measurement depth in the object E, of a portion corresponding to a data sequence increases. That is, for example, in the filter unit 48, first, five filters of which the central frequencies of the pass-bands are 16 MHz, 8 MHz, 4 MHz, 2 MHz, and 1 MHz are applied in parallel to the digital signals of a data sequence stored in the data area ADDR [1390:1050]. Subsequently, four filters of which the central frequencies of the pass-bands are 8 MHz, 4 MHz, 2 MHz, and 1 MHz are applied in parallel to the digital signals of a data sequence stored in the data area ADDR [1595:1391]. Subsequently, three filters of which the central frequencies of the pass-bands are 4 MHz, 2 MHz, and 1 MHz are applied in parallel to the digital signals of a data sequence stored in the data area ADDR [1765:1596].

Subsequently, three filters of which the central frequencies of the pass-bands are 4 MHz, 2 MHz, and 1 MHz are applied in parallel to the digital signals of a data sequence stored in the data area ADDR [1765:1596]. Subsequently, two filters of which the central frequencies of the pass-bands 2 MHz and 1 MHz are applied in parallel to the digital signals of a data sequence stored in the data area ADDR [1940:1766]. Finally, only a filter of which the central frequency of the pass-band is 1 MHz is applied to the digital signals of a data sequence stored in the data area ADDR [2047:1941]. That is, when filters to be used sequentially are selected in this manner, it is not necessary to apply the filter of which the central frequency of the pass-band is 16 MHz to the digital signals of a data sequence stored in the data area ADDR [2047:1391]. Thus, an unnecessary process does not occur and a filtering process in many frequency ranges can be performed efficiently.

In the description, an example in which one filter unit 48 and one multiplication unit 49 are disposed in the digital gain unit 40 has been illustrated. However, the present invention is not limited thereto, and a plurality of blocks of the filter unit 48 and the multiplication unit 49 may be prepared in one digital gain unit 40 so as to perform the following process. That is, the same digital signal may be input to the respective blocks, and the digital signal may be processed using different filter coefficients and digital gains in the respective blocks.

By doing so, the filtering process and the digital gain process can be performed in parallel in a plurality of frequency ranges, and the processing speed is improved.

The number of blocks of the filter unit 48 and the multiplication unit 49, disposed in one digital gain unit 40 may be the same as the number of frequency ranges which need to be processed by the object information acquiring apparatus and the process may be performed. The number of blocks prepared is appropriately determined within an allowable range of the system. In this case, the filter unit 48 to which a filter coefficient is allocated so that the pass-band is as high as 16 MHz processes a small number of digital signals and the process ends early. In this case, the filtering process itself may be stopped and the multiplication unit 49 may multiply the output signal of the filter unit 48 by 0 so that the filtering process stops substantially. When the filtering process is not stopped, unnecessary signals that do not contribute to photoacoustic image data are acquired. Thus, in this case, an unnecessary signal control unit (not illustrated) that controls the unnecessary signals to be not used by the reconstructing unit 5 may be provided.

Since the number of digital signals that include high-frequency components is small, a filter coefficient for high-frequency processing is supplied to the filter unit 48 and the filtering process ends in a short cycle when filtering for high-frequency processing is performed. In this case, the digital gain unit 40 supplies a filter coefficient for low-frequency processing to the filter unit 48 to which filter coefficients for high-frequency processing have been supplied. Moreover, digital signals including low-frequency components on which a filtering process has not been completed are processed to improve the processing speed. Specifically, the filter unit 48 is supplied with a filter coefficient for performing a 1-MHz filtering process from the digital gain generation unit 44 when the processing on the pass-band central frequency of 16 MHz ends. Moreover, a filtering process may be performed on the digital signals on which the 1-MHz filtering process has not been completed. However, the present invention is not limited thereto but a filter coefficient memory capable of temporarily storing the filter coefficients generated by the digital gain generation unit 44 may be provided and the filter coefficient may be appropriately supplied from the filter coefficient memory.

Even when only one block of the filter unit and the multiplication unit 49 is disposed in the digital gain unit 40, the processing efficiency can be improved based on the clock frequency supplied to the filter unit 48 and the multiplication unit 49. For example, the operating clock frequencies of the filter unit 48 and the multiplication unit 49 may be N times the clock cycle frequency used for inputting digital signals to the filter unit 48. Moreover, N filter coefficients and N digital gains may be applied to a set of digital signal groups (the number of signal groups depends on the number of filter taps) in one input clock cycle of digital data so that the effect equivalent to parallel processing to N blocks of the filter unit 48 and the multiplication unit 49 is obtained. Further, the output signals obtained through the filtering process and the digital gain process in the plurality of frequency ranges may be added and used for generation of photoacoustic image data. Moreover, a signal intensity checking circuit (not illustrated) may be provided at the output of the filter unit 48 of the digital gain unit 40 so that the multiplication unit 49 multiplies a valid digital gain value when a signal having an intensity equal to or higher than a certain threshold is detected. In this case, the multiplication unit 49 multiplies the signal having an intensity equal to or lower than a certain threshold by 0. By doing so, the multiplication unit 49 can supply digital signals in which noise components are cancelled to the reconstructing unit 5.

By employing the processing method described above, attenuation correction and light intensity correction can be performed ideally for signals having a wide frequency range generated inside the object E.

A case in which the attenuation in the media 1300 and 1400 can be ignored has been described. However, when a substance having such attenuation characteristics that cannot be ignored is used as the media 1300 and 1400, the digital gain pattern needs to be determined by taking the attenuation in the media 1300 and 1400 into consideration. Moreover, when the attenuation in the media 1300 and 1400 cannot be ignored, the degree of attenuation in the media 1300 and 1400, of the acoustic waves generated in the object E is different depending on the positional relation between the object E and the reception element 300. That is, the frequency characteristics of acoustic waves reaching the respective reception elements may be different for the respective reception elements. In this case, the type and the number of central frequencies of the pass-bands of filters applied in the filter unit 48 may be different for the respective reception elements 300.

Moreover, in the filter unit 48, the number of pass-bands applied to each reception element may be one or plural. Only one pass-band may be used for a filter and a plurality of pass-bands or a single pass-band may be switched according to the user's setting. For example, the filter unit 48 may use one band-pass filter and one low-pass filter. Alternatively, the filter unit 48 may extract a desired frequency range using a combination of one low-pass filter and one high-pass filter so that only one pass-band is eventually used. In this case, the digital gain may be set according to a desired frequency in the pass-band of a filter.

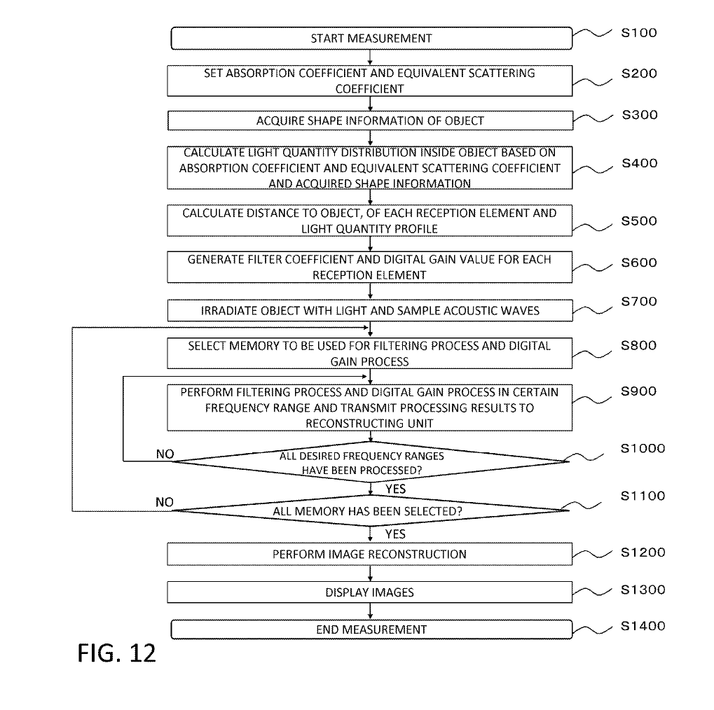

FIG. 12 is a flowchart illustrating the functions of the data acquisition unit according to Example 1, and a method for performing attenuation correction and light intensity correction on digital data will be described with reference to the flowchart. Measurement starts in step S100 and then the flow proceeds to step S200. In step S200, an operator inputs optical characteristic values of the object E to the input unit 1 so that the optical characteristic values of the object E are input to the apparatus 1000. Then, the flow proceeds to step S300.

In this example, the input unit 1 inputs an average absorption coefficient μ_{α}_{_}_{BG} and an equivalent scattering coefficient μₛ of the object E as the optical characteristic values in order to calculate a light intensity distribution in the object E. Further, the input unit 1 inputs an absorption coefficient μ_{α} (assumed value) of a light absorber (tumor or the like) in the object E, necessary for determining the digital gain value. That is, the input unit 1 inputs three parameters in total, including two absorption coefficients and one equivalent scattering coefficient. Moreover, the input unit 1 may input typical values of a living body and may input known statistical values corresponding to the characteristics (e.g. age) of the object E as the absorption coefficient μ_{α}_{_}_{BG}, the equivalent scattering coefficient μₛ, and the absorption coefficient μ_{α}. Alternatively, when the age or the gender is input, the values corresponding thereto may be automatically set and are used as the input values for the input unit 1. Alternatively, the input unit 1 may input measurement values measured by an apparatus provided separately from the apparatus 1000. Moreover, the input unit 1 may not necessarily input only one value but a range of values that the corresponding parameter takes may be input. The input unit 1 supplies the received absorption coefficient and equivalent scattering coefficient to the control unit 2, and the control unit 2 uses the supplied values as setting values.

In step S300, the object E is inserted in the shape holding portion 1100, and the acoustic matching material 800 is filled between the support 400 and the shape holding portion 1100 and between the shape holding portion 1100 and the object E. After that, in step S300, a shape imaging unit (not illustrated) acquires the shape information of the object E, and then, the flow proceeds to step S400. In step S300, two-dimensional shape information (sectional information of the object E) or three-dimensional shape information of the object E can be acquired. In step S400, a light intensity distribution inside the object E is calculated based on the optical characteristic values (the absorption coefficient μ_{α}_{_}_{BG} and the equivalent scattering coefficient μₛ) of the object E acquired in step S200 and the shape information of the object E acquired in step S300, and then, the flow proceeds to step S500.

In step S500, the distance between each of the plurality of reception elements and the object E is calculated based on the shape information of the object E acquired in step S300 and the light intensity distribution inside the object E acquired in step S400. Further, the light intensity profile of each reception element is also calculated, and then, the flow proceeds to step S600. In step S600, a pass-band (e.g. a cutoff frequency) of a filter to be applied to each reception element is specified, a filter coefficient (e.g. a relaxation period) and a digital gain pattern are calculated, and then, the flow proceeds to step S700. In this calculation, the absorption coefficient μ_{α} (assumed value) of the object E acquired in step S200, the distance between each reception element and the object E and the light intensity profile of each reception element acquired in step S500 are taken into consideration.

In step S700, the object E is irradiated with light from the optical system 200, acoustic waves are sampled by the data acquisition unit 4, and then, the flow proceeds to step S800. In step S800, one of the memories 46-1 to 46-8 is selected, and then, the flow proceeds to step S900. In step S900, data is read from an address of the selected memory, in which digital signals including signals having a desired frequency range are stored. Moreover, the digital gain unit 40 performs a filtering process, multiplies a digital gain value, and supplies a multiplication result to the reconstructing unit 5, and then, the flow proceeds to step S1000.

In step S1000, it is determined whether a digital gain process has been completed for all desired frequency ranges. When it is determined that the process has not been completed (that is, if a frequency range on which a digital gain process is to be performed remains), the flow proceeds to step S900. Moreover, when it is determined that the process has been completed (that is, when the digital gain process has been completed for all desired frequency ranges), the flow proceeds to step S1100. In step S1100, it is determined whether the digital gain process has been completed for a desired memory among the memories 46-1 to 46-8. When it is determined that the process has been completed (that is, when the digital gain process has been completed for the desired memory), the flow proceeds to step S1200. On the other hand, when it is determined that the process has not been completed (that is, if the memory 46 on which the digital gain process is to be performed remains), the flow proceeds to step S800.

In step S1200, the reconstructing unit 5 performs image reconstruction using the digital signals processed in steps S800 to S1100. In this way, the photoacoustic image data of the inside of the object is obtained. In step S1300, an image of the inside of the object is displayed on the display unit 6 based on the photoacoustic image data of the inside of the object obtained in step S1200. Digital signals of different frequency ranges may be individually subjected to image reconstruction so that items of image data of the plurality of frequency ranges are individually displayed, and items of image data of a plurality of frequency ranges may be combined and displayed in a superimposed manner. Alternatively digital signals of different frequency ranges may be added and subjected to image reconstruction and the reconstructed images may be displayed. In this case, frequency ranges may be displayed on an image using different colors so that the frequency ranges can be easily identified. Moreover, items of image data of a plurality of frequency ranges may be displayed individually, and in this case, the items of image data may be displayed in parallel. Alternatively, items of image data of a plurality of frequency ranges may be displayed so as to be switched every frame so that the frequency range of the displayed image data is different every frame. By doing so, such an effect that a plurality of items of image data are displayed in a superimposed manner can be obtained. In this case, the images to be displayed in a switched manner and the switching rate may be determined arbitrarily by the user. Moreover, a test mode for changing the switching display rate to determine an optimal rate may be provided. By employing such a display format, users can check individual blood vessels having different thicknesses on different images, for example, and the visibility of photoacoustic images is improved. Since the frequency characteristics of acoustic waves depend on the size of blood vessels (e.g. the thickness, the length, or the like of blood vessels), by allowing users to arbitrarily determine a frequency range of signals to be extracted, it is possible to generate and check photoacoustic images in which arbitrary blood vessels of different sizes are extracted. Moreover, by conducting diagnosis using photoacoustic images in which blood vessels having sizes within a certain range are extracted as well as the photoacoustic images in which blood vessels of various sizes are mixed, it is possible to improve the diagnosis accuracy.

The user may designate at least one frequencies of signals to be extracted. Moreover, the user may designate the shape and the size of a target object to be checked as a photoacoustic image. The frequency characteristics of acoustic waves may be different depending on whether the shape of a light absorber is spherical or tubular. In this case, for example, the control unit 2 may calculate the frequency characteristics of acoustic waves generated from the corresponding target object based on the shape and the size of the target object designated by the user and may generate a filter coefficient and a digital gain value used in the digital gain unit 40 based on the calculation result.

In step S1400, the measurement ends.

The apparatus 1000 acquires photoacoustic image data according to the operation flow described above. In this flow, although a case in which light is irradiated once has been illustrated, the number of light irradiations is not necessarily limited thereto. Reception signals obtained through a plurality of light irradiations may be integrated and image reconstruction may be performed using data having a high S/N ratio.

Moreover, in the present example, a method for determining a sampling frequency of acoustic waves in the ADCs 45-1 to 45-8 is also considered. That is, since the optical system 200 irradiates the entire object E with light, the ADCs 45-1 to 45-8 sample the acoustic waves generated near the surface of the object E. The acoustic waves generated near the surface of the object E may not have experienced attenuation inside the object E. Thus, if the attenuation in the media 1300 and 1400 can be ignored, acoustic waves having a very wide frequency range can reach the reception elements 300-1 to 300-8 depending on the size of the light absorber present near the surface of the object E. In this case, the sampling frequency may be determined by taking the reception band characteristics of the reception elements 300-1 to 300-8 and the range of resolutions of the light absorber that the user wants to observe (that is, the range of frequency characteristics of acoustic waves that the user is to acquire) into consideration.

Specifically, a frequency that is higher than twice the upper limit of the range of desired frequency characteristics (the upper limit of the reception band characteristics of the reception elements 300-1 to 300-8 or the upper limit of frequency characteristics of acoustic waves that the user is to acquire) may be determined as the sampling frequency. The sampling frequency may be set individually for the respective reception elements, and reception elements having adjacent setting values may be grouped and the same sampling frequency may be applied to reception elements in the same group. By doing so, it is possible to eliminate the difficulties of setting the sampling frequency individually. When the attenuation in the media 1300 and 1400 cannot be ignored, the ADCs 45-1 to 45-8 may perform the following process. That is, the sampling frequencies optimal for the respective reception elements 300-1 to 300-8 may be determined by taking the frequency characteristics of the acoustic waves generated near the surface of the object E and attenuated in the process of propagating through the media 1300 and 1400 into consideration. Moreover, the frequency characteristics of acoustic waves shift toward the low-frequency side with the elapse of reception time of the acoustic waves. This is because high-frequency components of acoustic waves are likely to decay in the object E, high-frequency components of acoustic waves generated in a deep portion of the object E decay more than compared to low-frequency components, and the low-frequency components are dominant in the acoustic waves. In this case, the sampling frequency may be controlled so as to decrease with the elapse of the reception time by taking such a shift of frequency characteristics into consideration.

The ADCs 45-1 to 45-8 allocate individual sampling frequencies to the respective reception elements 300-1 to 300-8 to acquire data. Thus, the time interval of the sampling data is different for the respective reception elements 300-1 to 300-8. For example, the following state can be considered. A state in which data having a time interval of 50 ns, obtained by sampling acoustic waves at 20 MHz in one of the reception elements 300-1 to 300-8 and data having a time interval of 25 ns, obtained by sampling acoustic waves at 40 MHz in another reception element are present together. In this case, when it is necessary to align the time intervals of the items of data between reception elements when the reconstructing unit performs image reconstruction, an interpolation process may be applied to the sampling data appropriately to generate items of data of which the time intervals are aligned. In the above example, an interpolation process may be applied to the data having the time interval of 50 ns, sampled at 20 MHz to generate data having a time interval of 25 ns and the generated data may be used for image reconstruction together with the data sampled at 40 MHz. Moreover, the apparatus 1000 may select sampling data of which the time interval fits a target resolution of the user among the items of data acquired by the reception elements 300-1 to 300-8 and use the selected data for image reconstruction. The data may be selected from a group of items of data sampled using the same reception element and may be selected from the respective reception elements, and both methods may be used.

As described above, in the apparatus 1000 of the present example, a setting pattern of the digital gain value (gain) multiplied with the digital signal can be different depending on a positional relation between each of the plurality of reception elements (acoustic wave detection elements) and the object E. By doing so, attenuation correction of acoustic waves for the respective reception elements can be optimized. Moreover, light intensity correction may be optimized according to a positional relation between a light intensity distribution inside the object E and the reception element. Moreover, it is possible to efficiently extract a desired frequency range from acoustic waves of a wide frequency range and to perform attenuation correction and light intensity correction. As a result, acoustic waves of a wide frequency range can be visualized satisfactorily.

### Example 2

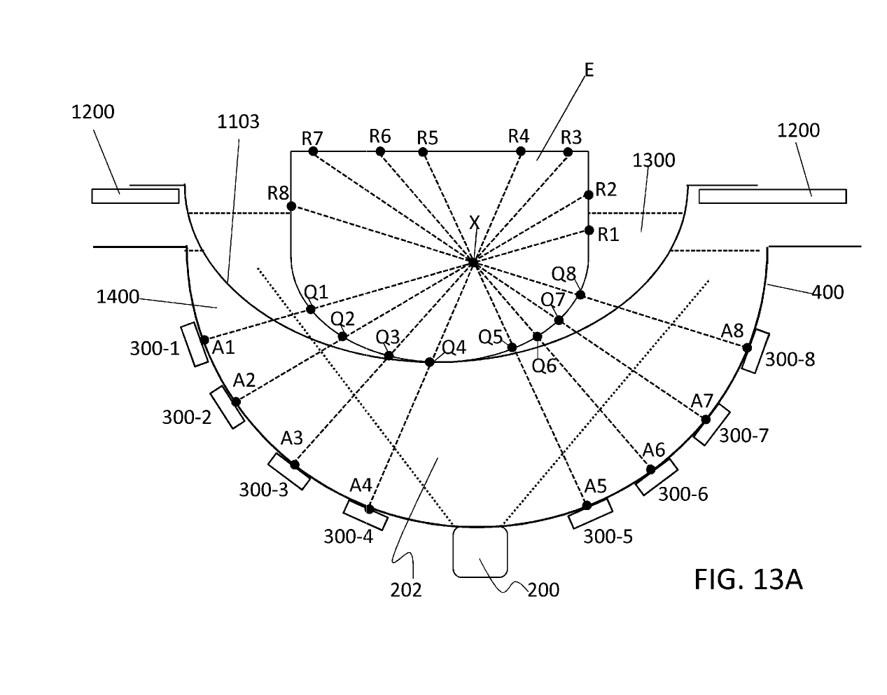

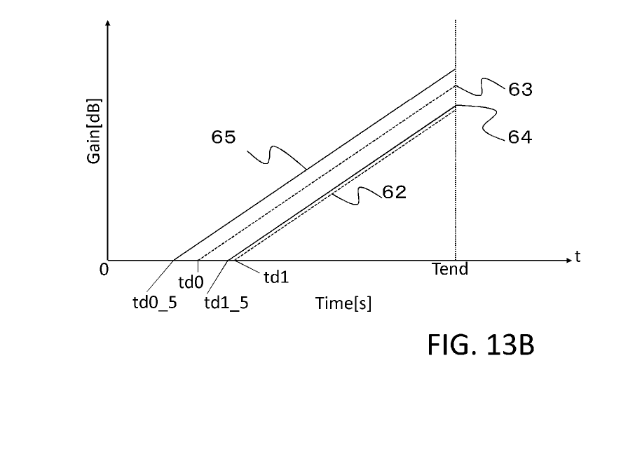

FIG. 13A is a schematic diagram illustrating Example 2 of the object information acquiring apparatus of the present invention, and the portions corresponding to those in the drawings described above will be denoted by the same reference numerals and the description thereof will be omitted unless necessary. The present example is different from Example 1 in that the support 400 moves and receives photoacoustic waves at a plurality of measurement positions. That is, the present example is different from Example 1 in that the positional relation between the object E and the support 400 and the positional relation between the object E and the reception elements 300-1 to 300-8 change according to the measurement position (a light irradiation position of the optical system 200). That is, the support 400 moves to another measurement position from the state illustrated in FIG. 7 and the positional relation between the object E and the support 400 is changed. In this case, the distance between the reception elements 300-1 to 300-8 and the object E in FIG. 7 is different from that of FIG. 13A. That is, when the measurement position changes, the distance between the reception elements 300-1 to 300-8 and the object E changes. In the present example, the digital gain generation unit 44 optimizes the digital gain pattern (gain) so that the individual digital gain patterns of the reception elements 300-1 to 300-8 can be different. In this case, the digital gain generation unit 44 generates different digital gain patterns (gains) for the respective measurement positions based on the distance between the reception elements 300-1 to 300-8 and the object E. The respective different digital gain patterns (gains) are multiplied with the digital signals output by the respective reception elements 300-1 to 300-8.

FIG. 13B is a diagram illustrating the relation between the sampling time and the digital gain pattern of digital signals. Hereinafter, a digital gain setting method will be described. FIG. 13B illustrates a case in which sampling starts at the light irradiation time t=0. FIG. 13B illustrates the correspondence in such a case between the sampling time of digital signals in the ADCs 45-1 to 45-8 and the digital gain pattern multiplied by the multiplication unit 49 for the digital signals sampled at a certain time. It is assumed that the filter unit 48 extracts signals having the central frequency of 1 MHz. A digital gain pattern 64 indicates a digital gain pattern multiplied with the digital signals based on the acoustic waves received by the reception element 300-4 in FIG. 13A. Moreover, a digital gain pattern 65 indicated a digital gain pattern multiplied with the digital signals based on the acoustic waves received by the reception element 300-1 in FIG. 13A. Here, it is assumed that time td1_5 is the time at which, when sampling starts at the light irradiation time t=0, acoustic waves generated at the point Q4 propagate through the segment Q4-A4 to reach the point A4. Moreover, it is assumed that time td0_5 is the time at which acoustic waves generated at the point Q1 propagate through the segment Q1-A1 to reach the point A1. That is, td0_5={(Length of Segment Q1-A1)/(Acoustic velocity in media 1300 and 1400)} and td1_5={(Length of Segment Q4-A4)/(Acoustic velocity in media 1300 and 1400)}. In this case, it is assumed that the acoustic velocity in the medium 1300 is the same as that of the medium 1400. When the acoustic velocities in the media 1300 and 1400 are different, the time td0_5 and td1_5 may be determined by taking the difference in the acoustic velocities into consideration.

When FIG. 7 and FIG. 13A are compared, since the distance between the reception element 300-4 and the object E is not changed too much, there is no great difference between the digital gain pattern 62 (illustrated commonly in FIG. 7 and FIG. 13A) and the digital gain pattern 64. On the other hand, the distance between the reception element 300-1 and the object E is changed and the length of the segment A1-Q1 is smaller than the length of the segment A1-F1. Thus, even when the digital signals of acoustic waves are received by the same reception element 300-1, the time at which a digital gain is applied in the case of FIG. 13A is earlier than that of FIG. 7. This state is depicted as a difference between the digital gain pattern 63 (illustrated commonly in FIG. 7 and FIG. 13A) and the digital gain pattern 65 in FIG. 13B. Moreover, a method for irradiating the object E with irradiation light 202 in FIG. 7 is different from that of FIG. 13A. Thus, for example, the light intensity profile on the segment F1-G1 in FIG. 7 may be different from the light intensity profile on the segment Q1-R1 in FIG. 13A. In other words, the light intensity profiles in acoustic wave generation areas corresponding to the reception elements 300-1 to 300-8 can change for the respective measurement positions. Thus, the multiplication unit 49 may change a gain value corresponding to light intensity correction for the respective measurement positions when light intensity correction is performed using the digital gain patterns.

Moreover, the shape of the object E may be detected once before the start of measurement as illustrated in step S300 of FIG. 12, and the shape may be detected whenever the measurement position of the receiving unit 320 is moved in a stepwise manner. When the shape is detected whenever the measurement position is moved, it is possible to set the digital gain pattern with higher accuracy because the digital gain pattern can be set according to the motions of the object E during the photoacoustic measurement. Moreover, in the present example, a method for determining the sampling frequency of each reception element is important. When the entire object E is irradiated with light, the sampling frequency may be determined in the manner described in Example 1. However, the present invention is not limited thereto but the shape may be detected while continuously moving the receiving unit 320.

As described above, in the present example, since the measurement position of the support 400 in relation to the object E changes, the positional relation between the object E and the reception element changes for the respective measurement positions. Thus, the setting pattern of the digital gain value multiplied with the digital signals changes for the respective measurement positions and the respective reception elements. By doing so, attenuation correction of acoustic waves can be optimized for the respective measurement positions and the respective reception elements. Moreover, light intensity correction can be optimized according to the positional relation between the light intensity distribution inside the object E and the reception element. As a result, acoustic waves of a wide frequency range can be visualized satisfactorily.

### Example 3

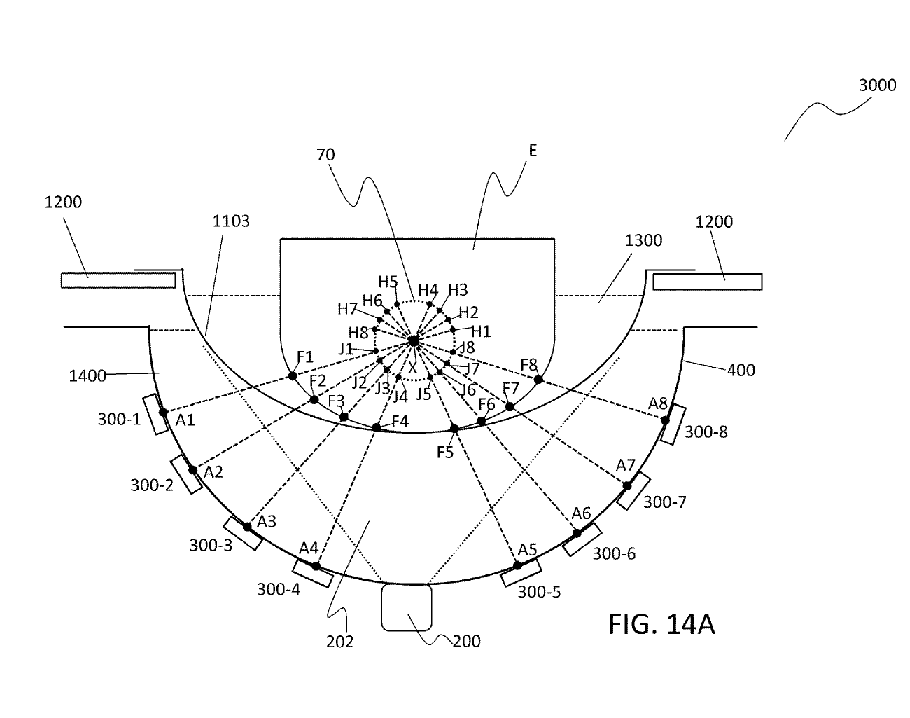

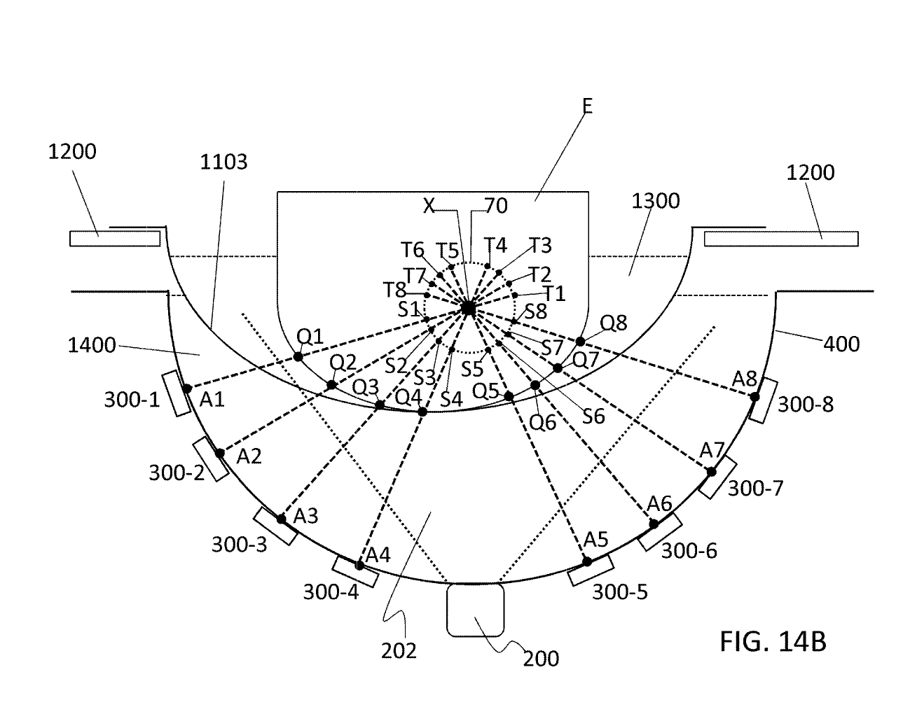

FIG. 14A is a schematic diagram illustrating Example 3 of the object information acquiring apparatus of the present invention, and the portions corresponding to those in the drawings described above will be denoted by the same reference numerals and the description thereof will be omitted unless necessary. The present example is different from Examples 1 and 2 in that the setting pattern of the digital gain value multiplied with the digital signals is changed by taking a data acquisition range as well as the positional relation between the object E and the reception element into consideration. An object information acquiring apparatus 3000 (hereinafter referred to simply as an “apparatus 3000”) of the present example samples acoustic waves originating from a high-resolution area 70 only when the entire object E is irradiated with light. Reception elements 300-n (n=1 to 8) selectively sample acoustic waves generated on a segment Jn-Hn. An irradiation unit may at least irradiate a data acquisition range with light having a sufficient intensity and does not necessarily irradiate the entire object E with light. In the present example, the irradiation unit may at least irradiate the high-resolution area 70 with light having a sufficient intensity. The high-resolution area is such an area to which the directions in which the reception sensitivities of the plurality of reception elements are highest are directed.

In the case of FIG. 14A, the digital gain generation unit 44 determines digital gain patterns (gains) based on the distance between each of the plurality of reception elements and the surface of the object E and the distance between the surface of the object E and a data acquisition area. The digital gain patterns (gains) are multiplied with the digital signals acquired for the respective reception elements 300-n (n=1 to 8). The respective values of the digital gain patterns (gains) can be different based on the distance between each of the plurality of reception elements and the surface of the object E and the distance between the surface of the object E and the data acquisition area. In this example, the surface of the object E and the data acquisition area are the high-resolution area 70, the distance between the reception element and the object E is the length of the segment An-Fn, and the distance between the surface of the object E and the data acquisition area is the length of the segment Fn-Jn. In the case of FIG. 14A, when the distance (the length of the segment An-Jn) between the reception element 300-n (n=1 to 8) and the high-resolution area 70 is known from the design of the apparatus 300, if the length of the segment An-Fn at the respective measurement positions can be known, the length of the segment Fn-Jn becomes obvious. That is, the length of the segment Fn-Jn is calculated by (Length of Segment Fn-Jn)=(Length of Segment An-Jn)−(Length of Segment An-Fn).

The apparatus 3000 mainly samples acoustic waves generated from the high-resolution area 70. Thus, when sampling starts at the light irradiation time t=0, the multiplication unit 49 multiplies signals sampled later than time t=ts0_n by a valid digital gain value. Here, ts0_n={(Length of Segment An-Fn)/(Acoustic velocity in Media 1300 and 1400)}+{(Length of Segment Fn-Jn)/(Acoustic velocity in Object E)}. Moreover, n is n=1 to 8 and corresponds to the reception element 300-n. In the case of FIG. 14A, multiplication start time (ts0_n) of a valid digital gain pattern needs to be adjusted by taking the time taken for acoustic waves to propagate the segment Fn-Jn as well as the time taken for acoustic waves to propagate through the segment An-Fn into consideration. Further, when a digital gain value is multiplied with the digital signals sampled at time t=ts0_n, the multiplication unit 49 adjust the digital gain value so that the initial value thereof is a gain value G0_n for correcting attenuation of acoustic waves having been generated at the point Jn and reached the point Fn.

FIG. 14B is a diagram illustrating a measurement state at another position of the receiving unit according to Example 3, and the portions corresponding to those in FIG. 14A will be denoted by the same reference numerals and the description thereof will be omitted unless necessary. In this case, the position of the high-resolution area 70 in the object E changes. Moreover, in this case, the distance between the reception elements 300-1 to 300-8 and the object E also changes. In this example, the apparatus 3000 mainly samples acoustic waves generated from the high-resolution area 70. Thus, when sampling starts at the light irradiation time t=0, the multiplication unit 49 multiplies signals sampled later than time t=ts1_n by a valid digital gain value. Here, ts1_n={(Length of Segment An-Qn)/(Acoustic velocity in Media 1300 and 1400)}+{(Length of Segment Qn-Sn)/(Acoustic velocity in Object E)}. Moreover, the multiplication unit 49 adjusts the multiplication start time (ts1_n) of a valid digital gain pattern by taking the time taken for acoustic waves to propagate through the segment Qn-Sn as well as the time taken for acoustic waves to propagate through the segment An-Qn into consideration. Further, a case in which the multiplication unit 49 multiplies the digital signals of which the sampling started at time t=ts1_n by a digital gain value will be considered. In this case, the digital gain generation unit 44 adjusts the digital gain value so that the initial value thereof is a gain value G1_n for correcting attenuation of acoustic waves having been generated at the point Sn and reached the point Qn.

In FIG. 14B, a segment Q1-S1 is a portion of a normal line of a reception surface of the reception element 300-1 which is a straight line extending in a direction in which the reception sensitivity of the reception element 300-1 is highest. Moreover, a segment Q8-S8 is a portion of a normal line of a reception surface of the reception element 300-8 which is a straight line extending in a direction in which the reception sensitivity of the reception element 300-8 is highest. The segment Q1-S1 is longer than the segment Q8-S8. That is, since acoustic waves generated on the segment S8-T8 propagate a shorter distance in the object E than acoustic waves generated on the segment S1-T1, high-frequency components are dominant in the acoustic waves when reached the reception element 300-8. On the other hand, since acoustic waves generated on the segment S1-T1 propagate a long distance in the object E, low-frequency components are dominant in the acoustic wave when reached the reception element 300-1. That is, in FIG. 14B, the type of the filter applied to the digital signals sampled by the reception element 300-1 can be different from that of the reception element 300-8. For example, the filter unit 48 may apply five filters of which the central frequencies of the pass-bands are 16 MHz, 8 MHz, 4 MHz, 2 MHz, and 1 MHz to the digital signals sampled by the reception element 300-8. On the other hand, since low-frequency components are dominant in the digital signals sampled by the reception element 300-1, the filter unit 48 may apply only three filters of which the central frequencies of the pass-bands are 4 MHz, 2 MHz, and 1 MHz to the digital signals.

By doing so, it is possible to eliminate an unnecessary process of applying a filter of which the central frequency of the pass-band is 16 MHz or 8 MHz to electrical signals based on acoustic waves that do not include 16-MHz or 8-MHz frequency components.

Alternatively, the filter unit 48 may apply a filter group of which the central frequency of the pass-band is as low as 4 MHz, 2 MHz, 1 MHz, 0.8 MHz, or 0.6 MHz to the digital signals sampled by the reception element 300-1 although the same number of filters is used. Which method will be used may be appropriately determined according to the frequency characteristics of signals that the user wants to extract.

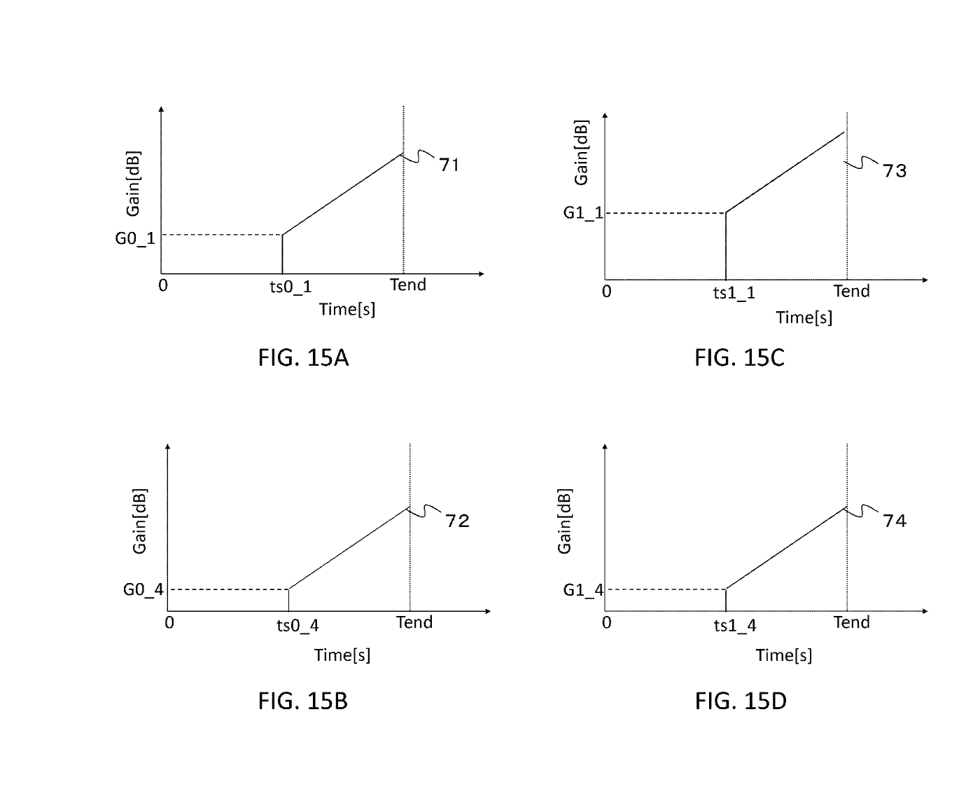

FIGS. 15A to 15D are diagrams illustrating the relation between the time and the digital gain pattern according to Example 3. FIG. 15A is a diagram illustrating the relation between the time and the digital gain pattern according to Example 3. That is, FIG. 15A illustrates a case in which sampling starts at the light irradiation time t=0. FIG. 15A illustrates the correspondence in such a case between the sampling time of digital signals in the ADCs 45-1 to 45-8 and the digital gain pattern multiplied by the multiplication unit 49 for the digital signals sampled at a certain time. In this case, it is assumed that the filter unit 48 extracts signals having the central frequency of 1 MHz. The gain value in FIG. 15A starts with a gain value G0_1 for the digital signals sampled at time t=ts0_1.

FIG. 15B is a diagram illustrating another relation between the time and the digital gain pattern according to Example 3. That is, FIG. 15B illustrates a case in which sampling starts at the light irradiation time t=0. FIG. 15B illustrates the correspondence in such a case between the sampling time of digital signals in the ADC 45-4 and the digital gain pattern multiplied by the multiplication unit 49 for the digital signals sampled by the reception element 300-4. In this case, it is assumed that the filter unit 48 extracts signals having the central frequency of 1 MHz. In the case of FIG. 15B, the gain value starts with an initial value G0_4 at time t=ts0_4. In FIG. 14A, since the length of the segment F4-J4 is shorter than the length of the segment F1-J1, the attenuation of acoustic waves having been generated at the point J4 and reached the point F4 is smaller than the attenuation of acoustic waves having been generated at the point J1 and reached the point F1. Thus, the initial value G0_4 of the attenuation correction corresponding to the reception element 300-4 is smaller than the initial digital gain value G0_1 of the attenuation correction corresponding to the reception element 300-1. The digital gain patterns illustrated in FIGS. 15A and 15B can be different depending on the central frequency of the signals extracted by the filter unit 48.

FIG. 15C is a diagram illustrating another relation between the time and the digital gain pattern according to Example 3. That is, FIG. 15C illustrates a case in which the light irradiation time is set to t=0. FIG. 15C illustrates the correspondence in such a case between the sampling time of digital signals in the ADC 45-1 and the digital gain pattern multiplied by the multiplication unit 49 for the digital signals sampled by the reception element 300-1.

FIG. 15D is a diagram illustrating another relation between the time and the digital gain pattern according to Example 3. That is, FIG. 15D illustrates a case in which the light irradiation time is set to t=0. FIG. 15D illustrates the correspondence in such a case between the sampling time of digital signals in the ADC 45-4 and the digital gain pattern multiplied by the multiplication unit 49 for the digital signals sampled by the reception element 300-4. In this case, it is assumed that the filter unit 48 extracts signals having the central frequency of 1 MHz. The gain value in FIG. 15C starts with an initial value G1_1 at time t=ts1_1, and the gain value in FIG. 15D starts with an initial value G1_4 at time ts1_4. In FIG. 14B, the acoustic waves generated at the point S1 are attenuated more than the acoustic waves having the same frequency, generated at the point S4. This is because the length of the segment Q1-S1 is smaller than the length of the segment Q4-S4. Thus, the initial value G1_1 is larger than the initial value G1_4. For example, when the size of the high-resolution area 70 changes or another data acquisition area is to be set, the multiplication unit 49 may change the gain value to be applied according to the data acquisition area.

As described above, in the present example, the digital gain generation unit 44 changes the setting pattern of the digital gain value to be multiplied with the digital signal according to the positional relation between the object E and the reception element and the data acquisition area. By doing so, it is possible to optimize the attenuation correction of acoustic waves for the respective reception elements. Moreover, the filter coefficient generation unit 43 of the present example may set the filter coefficient to be supplied to the filter unit 48 so as to be different for the respective reception element according to the positional relation between the object E and the reception element and the data acquisition area.

In the present example, the frequency characteristics of the acoustic waves reaching the respective reception elements can be different depending on the positional relation between the object E and the reception element and the data acquisition area in the object E. Thus, the filter unit 48 performs processing by changing the filter coefficient to be used for the respective reception elements. By doing so, it is possible to optimize the attenuation correction of acoustic waves for the respective reception elements and to eliminate an unnecessary filtering process. Moreover, the light intensity profile on the segment Sn-Tn (n=1 to 8) can be different for the respective measurement positions and the respective reception elements since a mode in which the irradiation light 202 strikes the high-resolution area 70 is different for the respective measurement positions. In the present example, when light intensity correction is performed together with the attenuation correction, the digital gain generation unit 44 may set the gain value by taking the difference in the light intensity profile in the data acquisition area for the respective reception elements and the respective measurement positions into consideration. Further, when digital data is supplied from the memory unit 46 to the digital gain unit 40, the apparatus 3000 may selectively read the digital data on the segment Sn-Tn (n=1 to 8) and supply the selected digital data to the digital gain unit 40. In this case, the multiplication unit 49 may prepare a number of gain patterns to be multiplied with the output from the filter unit 48 so as to correspond to the segment Sn-Tn (n=1 to 8). In the present example, the upper limit of the frequency characteristics of acoustic waves reaching the reception elements 300-1 to 300-8 depends on the position of the data acquisition area in the object E. The distance of the segment Fn-Jn in the case of FIG. 14A and the distance of the segment Sn-Qn in the case of FIG. 14B affect the upper limit of the frequency characteristics of acoustic waves reaching the reception element 300-n. Thus, in the present example, the sampling frequency may be determined depending on the position of the data acquisition area in the object E. When the frequency characteristics of acoustic waves that the user wants to acquire are lower than the upper limit of the frequency characteristics of acoustic waves reaching the reception element 300-n, the sampling frequency may be determined according to the lower frequency characteristics. In addition to these matters, the matters that need to be considered in relation to the sampling frequency are the same as those of Example 1 or 2.

The apparatus 3000 can visualize acoustic waves of a wide frequency range satisfactorily by performing the above-described processes.

### Example 4

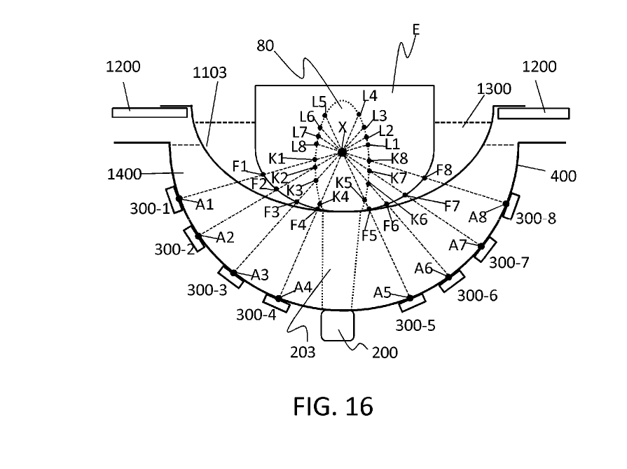

FIG. 16 is a schematic diagram illustrating Example 4 of the object information acquiring apparatus of the present invention, and the portions corresponding to those in the drawings described above will be denoted by the same reference numerals and the description thereof will be omitted unless necessary. The present example is different from Examples 1 to 3 in that the setting pattern of the digital gain values to be multiplied with the digital signals is changed based on a light distribution range in the object E as well as the positional relation between the object E and the reception element. FIG. 16 illustrates a case in which a portion of the object E is irradiated with light and the reception element 300-n (n=1 to 8) receives acoustic waves generated on a segment Kn-Ln. In this case, the digital gain generation unit 44 generates the digital gain pattern to be multiplied with the digital signals of each reception element based on the distance between the reception element and the object E and the distance between the surface of the object E and a light intensity distribution area 80. The distance between the reception element and the object E is the length of the segment An-Fn, and the distance between the surface of the object E and the light intensity distribution area 80 is the length of the segment Fn-Kn. In this case, the digital gain generation unit 44 may generate a digital gain pattern that can correct the distance-dependent attenuation inside the object and correct the light intensity distribution. The light intensity distribution area 80 may be stored in the apparatus by calculating the shape of the area and the distance to the respective acoustic wave detection elements through computer-based simulation before actual photoacoustic measurement is performed. Moreover, when a photoacoustic measurement method for irradiating the object E with light while moving the optical system 200 and the support 400 is employed, the light intensity distribution area 80 at each light irradiation position may be calculated and recorded in a memory (not illustrated) for storing the calculation result of the light intensity distribution area 80.

In a photoacoustic imaging method which is one of object information acquisition methods, since acoustic waves are not generated from an area in the object E in which light does not strike, sampling data corresponding to the portion in which acoustic waves are not generated is basically not significant data. Thus, it is not useful to consider the gain value for such data. That is, the digital gain generation unit 44 does not necessarily need to generate a gain value for sampling data corresponding to the portion between the surface of the object E and the light intensity distribution area 80 (that is, the sampling data corresponding to the segment Fn-Kn). Thus, the gain value may not be generated for the sampling data corresponding to the segment Fn-Kn. In the case of FIG. 16, the digital gain generation unit 44 sets a gain value for acoustic waves generated inside the light intensity distribution area 80. Moreover, when sampling starts at the light irradiation time t=0, the multiplication unit 49 multiplies the digital gain value with signals sampled later than time t=tl0_n. Here, tl0_n={(Length of Segment An-Fn)/(Acoustic velocity in Media 1300 and 1400)}+{(Length of Segment Fn-Kn)/(Acoustic velocity in Object E)}. Moreover, the digital gain generation unit 44 adjusts the initial value of the digital gain value by an amount corresponding to the time taken for the acoustic waves to propagate the length of the segment Fn-Kn. This means that the initial value of the digital gain value is set according to the same idea as described in Example 3.

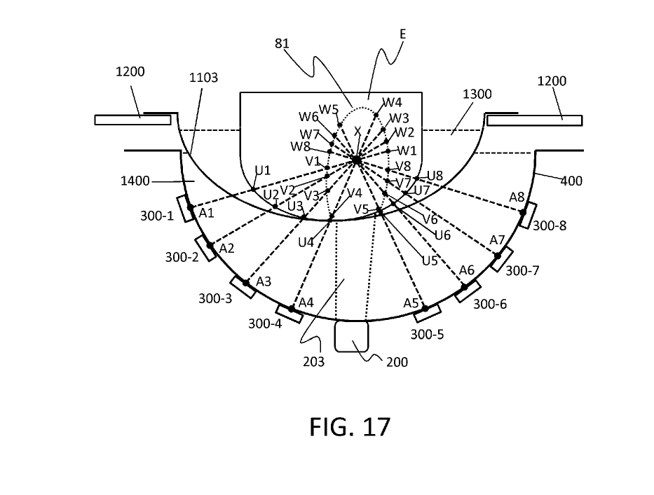

FIG. 17 is a diagram illustrating another measurement state of the object information acquiring apparatus of Example 4. That is, the positional relation between the support 400 and the object E in FIG. 17 is different from that of FIG. 16. In this case, the position at which a light intensity distribution area 81 is present in the object E is different from the position at which the light intensity distribution area 80 is present in the object E. The shape of the light intensity distribution area 81 is different from the shape of the light intensity distribution area 80. This is because the shape of a portion in which irradiation light 203 strikes the object E in FIG. 16 is different from that of FIG. 17. The distance between the reception elements 300-1 to 300-8 and the object E in FIG. 16 is different from that of FIG. 17. In the case of FIG. 17, the digital gain generation unit 44 sets the gain for acoustic waves generated in the light intensity distribution area 81. When sampling starts at the light irradiation time t=0, the multiplication unit 49 multiplies signals sampled later than time t=tl1_n by a digital gain value. Here, tl1_n={(Length of Segment An-Un)/(Acoustic velocity in Media 1300 and 1400)}+{(Length of Segment Un-Vn)/(Acoustic velocity in Object E)}. In the case of FIG. 17, the digital gain generation unit 44 adjusts the initial value of the digital gain value by an amount corresponding to the time taken for acoustic waves to propagate the segment Un-Vn. The digital gain generation unit 44 may set the initial value according to the same method as used in Example 3.

As described above, in the present example, the digital gain value of each reception element is set according to a change in the light intensity distribution area at each measurement position. In this case, similarly to Example 3, the frequency characteristics of acoustic waves reaching the reception element 300-n can be different depending on the distance between the light intensity distribution area and the object E (the length of the segment Fn-Kn in FIG. 16 and the length of the segment Un-Vn in FIG. 17 where n=1 to 8). Thus, the filter unit 48 sets the filter characteristics for processing received digital signals so as to be different for the respective measurement positions or the respective reception elements.

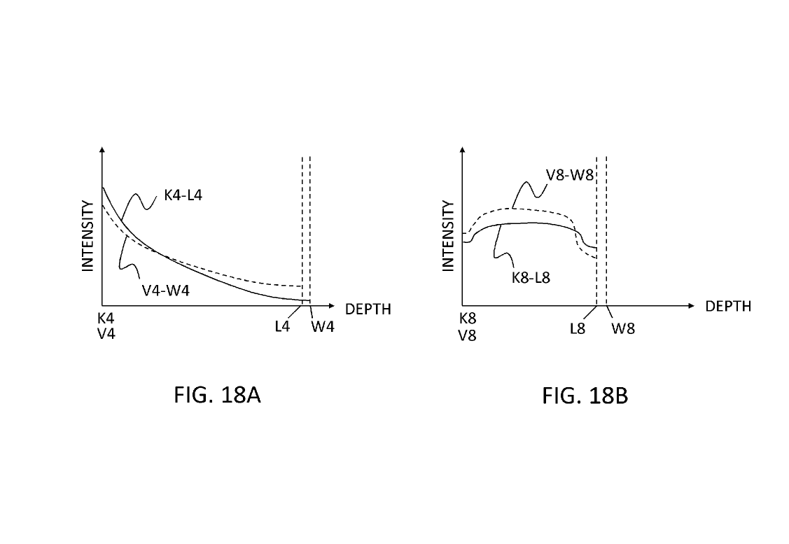

FIGS. 18A and 18B are diagrams illustrating the light intensity profile. In the light intensity profiles on the segment Kn-Ln (n=1 to 8) and the segment Vn-Wn (n=1 to 8), the light intensity distribution states of the light intensity distribution areas 80 and 81 are different for respective measurement positions. Thus, as illustrated in FIGS. 18A and 18B, the light intensity distribution states can be different even when n is the same. In the present example, when the light intensity correction is performed together with the attenuation correction, the digital gain generation unit may generate the gain value by taking the change in the light intensity profile of the light intensity distribution area for the respective reception elements or the measurement positions into consideration. Further, the digital gain unit 40 may perform the following process when digital data is supplied from the memory unit 46 to the digital gain unit 40. That is, the digital gain unit may selectively read digital data based on the acoustic waves generated inside the light intensity distribution area (for example, the segment Kn-Ln (FIG. 16) and the segment Vn-Wn (FIG. 17)) from the memory unit 46. Moreover, the digital gain unit 40 may input the read data thereto. In this case, the digital gain generation unit 44 may generate a number of gain patterns to be multiplied by the multiplication unit 49 with the output from the filter unit 48 so as to correspond to the light intensity distribution area.

In the present example, the method for determining the sampling frequencies of acoustic waves in the ADCs 45-1 to 45-8 is different from that of Examples 1 to 3. In the present example, the upper limit of the frequency characteristics of acoustic waves reaching the reception elements 300-1 to 300-8 depends on the position of the light intensity distribution area in the object E. That is, the distance of the straight line from the point Fn to the point Kn in FIG. 16 and the distance of the straight line from the point Un to the point Vn in FIG. 17 affect the upper limit of the frequency characteristics of acoustic waves reaching the reception element 300-n. Thus, in the present example, the sampling frequencies of the acoustic waves in the ADCs 45-1 to 45-8 may be determined based on the position of the data acquisition area in the object E. When the frequency characteristics of acoustic waves that the user wants to acquire are lower than the upper limit of the frequency characteristics of acoustic waves reaching the reception element 300-n, the sampling frequency may be determined according to the lower frequency characteristics. The matters that need to be considered in relation to the sampling frequency are the same as those of Examples 1 to 3.

As described above, in the present example, the frequency characteristics of the acoustic waves reaching the respective reception elements can be different depending on the positional relation between the object E and the reception element and the light intensity distribution area. Thus, the filter coefficient generation unit 43 may generate different filter coefficients for the respective reception elements and the filter unit 48 may perform filtering on the data input from the memory unit 46 using the different filter coefficients. By doing so, it is possible to optimize the attenuation correction of acoustic waves for the respective reception elements. Further, it is possible to eliminate an unnecessary filtering process. Further, it is possible to visualize acoustic waves of a wide frequency range satisfactorily.

As described above, the object information acquiring apparatus of Examples 1 to 4 optimizes the characteristics of filters to be applied to the respective reception elements based on the positional relation between the reception element and the data acquisition area when acoustic waves generated from the data acquisition area are acquired. Further, the apparatus extracts signal components of a specific frequency range from digital signals based on the acoustic waves and optimizes a digital gain pattern for correcting attenuation of the extracted signals for the respective reception elements. The data acquisition area is the object shape itself, a visualization area set in the object, a light intensity distribution area in the object, and an area on which the directions in which the reception sensitivities of all reception elements are highest concentrate. The frequency of acoustic waves generated by a light absorber depends also on a pulse width of light irradiated to the object E. For example, when the pulse width increases, the frequency characteristics of the acoustic waves generated by the light absorber shift toward the low-frequency side. Thus, the pulse width of the irradiated light may be taken into consideration in the digital gain pattern.

### Other Embodiments

The digital gain patterns are illustrated in FIG. 9, FIG. 13B, and FIGS. 15A to 15D. The gain value before a valid digital gain value is applied may be set to an arbitrary value. Moreover, although it has been described that eight reception elements 300 are provided in the support 400, the number is not necessarily limited thereto but can be changed appropriately as necessary. When there is one reception element 300, the reception element 300 may be moved to a plurality of measurement positions, for example, to perform photoacoustic measurement of the object E. By doing so, the same effect as the case of acquiring acoustic waves using a plurality of reception elements can be obtained.

Moreover, the arrangement of the reception elements 300 provided in the support 400 is not limited to the illustrated form, and various other arrangements may be used. Moreover, the digital gain pattern, the filter coefficient, and the sampling frequency may not be different for the respective reception elements 300. Reception elements of which the setting contents are similar among the reception elements 300 may be grouped into a plurality of groups, and the same digital gain pattern and the same filter coefficient may be set for each of the individual groups. By doing so, it is possible to eliminate the difficulties of setting the pattern and the coefficient individually.

Moreover, the same grouping method or different grouping methods may be used for setting the digital gain pattern, the filter coefficient, and the sampling frequency. Moreover, the grouping method may be changed for the same measurement position and may be changed with a change in measurement conditions such as light irradiation conditions and may be changed whenever the measurement position changes. The digital gain pattern, the filter coefficient, and the sampling frequency corresponding to the respective reception elements 300 may not necessarily be changed when the measurement position changes, but the same settings may be used even when the measurement position changes.

The digital gain pattern, the filter coefficient, and the sampling frequency may be set in the following manner when the same setting is performed at a plurality of measurement positions. That is, the plurality of measurement positions may be different for the settings of the digital gain pattern, the filter coefficient, and the sampling frequency. For example, the movable area of the support 400 may be divided into a plurality of areas, and the digital gain pattern, the filter coefficient, and the sampling frequency are set in the same manner in each divided movable area (including at least one measurement positions). Moreover, the movable area may be divided in a different manner for the settings of the digital gain pattern, the filter coefficient, and the sampling frequency. It is not always necessary to set a plurality of digital gain patterns and a plurality of filter coefficients to be applied to each reception element 300 but only one digital gain pattern and only one filter coefficient may be set to be applied to each reception element.

Moreover, the number of digital gain patterns and the number of filter coefficients to be applied to the reception elements 300 may be different for the respective reception elements. In attenuation correction of the respective reception elements 300, the digital gain for attenuation correction may be set by taking a positional shift between a reference point provided within an object shape when the object E is projected on the XY plane in FIG. 1B and a measurement position on the XY plane of the support 400 into consideration. Further, although it has been described that the attenuation correction is performed up to an assumed initial acoustic pressure level, the acoustic pressure level to which attenuation is corrected may be set by the users for the respective reception elements, frequencies, and imaging depths. Moreover, the processes performed by the digital gain unit 40 of the data acquisition unit 4 may be performed by hardware such as FPGA (field-programmable gate array) or ASIC (application specific integrated circuit) or may be performed by software using GPU (graphics processing unit), DSP (digital signal processor), or CPU (central processing unit). Further, when a TGC process is performed on the electrical signals output by the reception element 300 during the sampling, the digital gain patterns for attenuation correction and light intensity correction may be adjusted by taking the gain of the TGC into consideration. Here, “TGC” is an abbreviation of time gain control.

The digital gain patterns illustrated in the examples above are examples only and are limited thereto.

In the above-described examples, although an example in which the plurality of reception elements 300 of the probe unit 3 are arranged on the support 400 having an approximately hemispherical shape has been illustrated, the configuration of the probe unit 3 is not limited thereto. The probe in which the plurality of reception elements 300 are arranged may have various other shapes including a linear shape, a 1.5D shape, a 2D shape, a convex shape, and an arc-shape. An imaging method in which photoacoustic measurement is performed by pressing the object E using parallel flat plates and moving the probe on the parallel flat plates and an imaging method in which photoacoustic measurement is performed by bringing the probe into direct contact with the object E and moving the probe on the object E with the hands of a person or with the aid of a moving mechanism may be employed. The present invention can be also applied to these cases.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-008892, filed on Jan. 20, 2015, which is hereby incorporated by reference herein in its entirety.

What is claimed is:

1. An object information acquiring apparatus comprising:
an irradiation unit configured to irradiate an object with light;
a receiving unit including a plurality of acoustic wave detection elements configured to receive acoustic waves generated from the object by irradiation with the light by the irradiation unit, and to output analog signals;
a conversion unit configured to perform analog-to-digital conversion on the analog signals output from the plurality of acoustic wave detection elements to generate a plurality of first digital signals;
a memory configured to store the plurality of first digital signals;
a correction unit configured to generate a plurality of second digital signals by correcting the plurality of first digital signals stored in the memory using a different frequency filter in accordance with a positional relation between a position in which characteristic information of the object is acquired and each of the plurality of acoustic wave detection elements; and
an acquiring unit configured to acquire the characteristic information of the object based on the plurality of second digital signals.

2. The object information acquiring apparatus according to claim 1, wherein
the area in which the characteristic information of the object is acquired is a region of interest set to the object.

3. The object information acquiring apparatus according to claim 1, wherein
the correction unit includes:
an extraction processing unit configured to extract signal components of a predetermined frequency range of the plurality of first digital signals based on the positional relation; and
a multiplication unit configured to generate the plurality of second digital signals by correcting the extracted signal components of the predetermined frequency range of the plurality of first digital signals using the frequency filter.

4. The object information acquiring apparatus according to claim 3, wherein
the frequency filter has a gain which is larger as a central frequency of the predetermined frequency range is larger.

5. The object information acquiring apparatus according to claim 3, wherein
the extraction processing unit is a filter that cuts off signal components outside the predetermined frequency range of the plurality of first digital signals based on the positional relation.

6. The object information acquiring apparatus according to claim 5, wherein
the filter is at least one of a band-pass filter, a high-pass filter, and a low-pass filter.

7. The object information acquiring apparatus according to claim 1, wherein
the receiving unit supports the plurality of acoustic wave detection elements so that directions in which reception sensitivities of the acoustic wave detection elements are highest concentrate.

8. The object information acquiring apparatus according to claim 7, wherein
the receiving unit supports the plurality of acoustic wave detection elements along an approximately hemispherical shape.

9. The object information acquiring apparatus according to claim 1, further comprising a moving mechanism configured to change a positional relation between the receiving unit and the object, wherein
the irradiation unit irradiates the light when the positional relation is a predetermined positional relation.

10. An object information acquisition method for acquiring characteristic information of an object based on a plurality of first signals obtained and stored by receiving, using a plurality of acoustic wave detection elements, acoustic waves generated from the object when irradiated with light, the method comprising:
a first step of generating a plurality of second signals by correcting the plurality of first signals using a different frequency filter in accordance with a positional relation between a position in which characteristic information of the object is acquired and each of the plurality of acoustic wave detection elements; and
a second step of acquiring the characteristic information of the object based on the plurality of second signals.

11. A non-transitory computer readable storing medium recording a computer program for causing a computer to perform an object information acquisition method for acquiring characteristic information of an object based on a plurality of first signals obtained and stored by receiving, using a plurality of acoustic wave detection elements, acoustic waves generated from the object when irradiated with light, the method comprising:
a first step of generating a plurality of second signals by correcting the plurality of first signals using a different frequency filter in accordance with a positional relation between a position in which characteristic information of the object is acquired and each of the plurality of acoustic wave detection elements; and
a second step of acquiring the characteristic information of the object based on the plurality of second signals.
